(19) United States
(12) Patent Application Publication
(10) Pub. No.: US 2009/0319029 A1
(43) Pub. Date: Dec. 24, 2009

(54) DOCKING APPARATUS AND METHODS OF USE

(75) Inventors: Michael A. Evans, Palo Alto, CA (US); Ivan Tzvetanov, Palo Alto, CA (US); Steven L. Herbowy, Palo Alto, CA (US); Raj P. Ganpath, Mountain View, CA (US); Amy Lee, Sunnyvale, CA (US); Anant Kumar, San Jose, CA (US); Gwendolyn A. Watanabe, Sunnyvale, CA (US); K.T. Venkateswara Rao, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Nellix, Inc., Palo Alto, CA (US)

(21) Appl. No.: 12/478,208

(22) Filed: Jun. 4, 2009

Related U.S. Application Data
(60) Provisional application No. 61/058,695, filed on Jun. 4, 2008.

(51) Int. Cl. A61F 2/06; A61M 29/00

(52) U.S. Cl. 623/1.35; 606/192

(57) ABSTRACT

A system for treating an aneurysm in a blood vessel comprises a docking scaffold having with upstream and downstream ends, and a central passageway therebetween. The upstream end engages the blood vessel upstream of the aneurysm. A portion of a first and second scaffolds are slidably received in the central passageway such that an outside surface of the first and second scaffolds engage an inside surface of the docking scaffold. A double-walled filling structure has outer and inner walls and the filling structure is adapted to be filled with a hardenable fluid filling medium so that the outer wall conforms to an inside surface of the aneurysm and the inner wall forms a substantially tubular lumen to provide a path for blood flow therethrough. The double-walled filling structure is coupled with at least one of the first and second leg scaffolds in expanded configuration.

### CROSS-REFERENCES TO RELATED APPLICATIONS

The present application is a non-provisional of, and claims the benefit of priority under 35 U.S.C. § 119(e) of U.S. Provisional Application No. 61/058,695 (Attorney Docket No. 025925-002800US) filed Jun. 4, 2008, the entire contents of which are incorporated herein by reference.

### STATEMENT AS TO RIGHTS TO INVENTIONS MADE UNDER FEDERALLY SPONSORED RESEARCH AND DEVELOPMENT

Not ApplicableNOT APPLICABLE

### REFERENCE TO A “SEQUENCE LISTING,” A TABLE, OR A COMPUTER PROGRAM LISTING APPENDIX SUBMITTED ON A COMPACT DISK

Not ApplicableNOT APPLICABLE

### BACKGROUND OF THE INVENTION

1. Field of the Invention

The present invention relates generally to medical systems and methods for treatment. More particularly, the present invention relates to systems and methods for treating aneurysms.

Aneurysms are enlargements or “bulges” in blood vessels which are often prone to rupture and which therefore present a serious risk to the patient. Aneurysms may occur in any blood vessel but are of particular concern when they occur in the cerebral vasculature or the patient's aorta.

The present invention is particularly concerned with aneurysms occurring in the aorta, particularly those referred to as aortic aneurysms. Abdominal aortic aneurysms (AAA's) are classified based on their location within the aorta as well as their shape and complexity. Aneurysms which are found below the renal arteries are referred to as infrarenal abdominal aortic aneurysms. Suprarenal abdominal aortic aneurysms occur above the renal arteries, while thoracic aortic aneurysms (TAA's) occur in the ascending, transverse, or descending part of the upper aorta.

Infrarenal aneurysms are the most common, representing about eighty percent (80%) of all aortic aneurysms. Suprarenal aneurysms are less common, representing about 20% of the aortic aneurysms. Thoracic aortic aneurysms are the least common and often the most difficult to treat.

The most common form of aneurysm is “fusiform,” where the enlargement extends about the entire aortic circumference. Less commonly, the aneurysms may be characterized by a bulge on one side of the blood vessel attached at a narrow neck. Thoracic aortic aneurysms are often dissecting aneurysms caused by hemorrhagic separation in the aortic wall, usually within the medial layer. The most common treatment for each of these types and forms of aneurysm is open surgical repair. Open surgical repair is quite successful in patients who are otherwise reasonably healthy and free from significant co-morbidities. Such open surgical procedures may be problematic, however, since access to the abdominal and thoracic aortas is difficult to obtain and because the aorta must be clamped off, placing significant strain on the patient's heart.

Over the past decade, endoluminal grafts have come into widespread use for the treatment of aortic aneurysm in patients who cannot undergo open surgical procedures. In general, endoluminal repairs access the aneurysm “endoluminally” through either or both iliac arteries in the groin. The grafts, which typically have been fabric or membrane tubes supported and attached by various stent structures, are then implanted, typically requiring several pieces or modules to be assembled in situ. Successful endoluminal procedures have a much shorter recovery period than open surgical procedures.

Present endoluminal aortic aneurysm repairs, however, suffer from a number of limitations. For example, a significant number of endoluminal repair patients experience leakage at the proximal juncture (attachment point closest to the heart) within two years of the initial repair procedure. While such leaks can often be fixed by further endoluminal procedures, the need to have such follow-up treatments significantly increases cost and is certainly undesirable for the patient. A less common but more serious problem has been graft migration. In instances where the graft migrates or slips from its intended position, open surgical repair is required. This is a particular problem since the patients receiving the endoluminal grafts are often those who are not considered to be good surgical candidates.

Further shortcomings of the present endoluminal graft systems relate to both deployment and configuration. For example, many of the commercially available endovascular systems are too large (above 12F) for percutaneous introduction. Moreover, current devices often have an annular support frame that is stiff and difficult to deliver as well as unsuitable for treating many geometrically complex aneurysms, particularly infrarenal aneurysms with little space between the renal arteries and the upper end of the aneurysm, referred to as short-neck or no-neck aneurysms. Aneurysms having torturous geometries, are also difficult to treat.

For these reasons, it would be desirable to provide improved methods and systems for the endoluminal and minimally invasive treatment of aortic aneurysms. In particular, it would be desirable to provide prostheses with better sealing and minimal or no endoleaks. It would also be desirable to provide prostheses which resist migration, which are flexible, relatively easy to deploy, use standardize components and/or a modular design that can treat many if not all aneurismal configurations, including short-neck and no-neck aneurysms as well as those with highly irregular and asymmetric geometries. It would be further desirable to provide systems and methods which are compatible with current designs for endoluminal stents and grafts, including single lumen stents and grafts, bifurcated stents and grafts, parallel stents and grafts, as well as with double-walled filling structures which are the subject of the commonly owned, copending applications described below. The systems and methods would preferably be deployable with the stents and grafts at the time the stents and grafts are initially placed. Additionally, it would be desirable to provide systems and methods for repairing previously implanted aortic stents and grafts, either endoluminally or percutaneously. At least some of these objectives will be met by the inventions described hereinbelow.

2. Description of the Background Art

U.S. Patent Publication No. 2006/0025853 describes a double-walled filling structure for treating aortic and other aneurysms. Copending, commonly owned U.S. Patent Publication No. 2006/0212112, describes the use of liners and extenders to anchor and seal such double-walled filling structures within the aorta. The full disclosures of both these publications are incorporated herein by reference. PCT Publication No. WO 01/21108 describes expandable implants attached to a central graft for filling aortic aneurysms. See also U.S. Pat. Nos. 5,330,528; 5,534,024; 5,843,160; 6,168,592; 6,190,402; 6,312,462; 6,312,463; U.S. Patent Publications 2002/0045848; 2003/0014075; 2004/0204755; 2005/0004660; and PCT Publication No. WO 02/102282.

### BRIEF SUMMARY OF THE INVENTION

The present invention provides systems and methods for the treatment of aneurysms, particularly aortic aneurysms including both abdominal aortic aneurysms (AAA) and thoracic aortic aneurysms (TAA).

In a first aspect of the present invention a system for treating an aneurysm in a blood vessel comprises a docking scaffold radially expandable from a contracted configuration to an expanded configuration and having an upstream end, a downstream end and a central passageway therebetween. In the expanded configuration the upstream end engages a portion of the blood vessel upstream of the aneurysm. The system also comprises a first leg scaffold that is radially expandable from a contracted configuration to an expanded configuration and a portion of the first leg scaffold is slidably received in the central passageway such that an outside surface of the first leg scaffold in the expanded configuration engages an inside surface of the docking scaffold. The system also comprises a second leg scaffold radially expandable from a contracted configuration to an expanded configuration, and a portion of the second leg scaffold is slidably received in the central passageway such that an outside surface of the second leg scaffold in the expanded configuration engages an inside surface of the docking scaffold. A first double-walled filling structure is coupled with at least one of the leg scaffolds in the expanded configuration. The filling structure has an outer wall and an inner wall, and the filling structure is adapted to be filled with a hardenable fluid filling medium so that the outer wall conforms to an inside surface of the aneurysm and the inner wall forms a first substantially tubular lumen to provide a path for blood flow therethrough.

The hardenable filling material may comprise a polymer and the blood vessel may be an aorta. Often, the aneurysm is an abdominal aortic aneurysm. The system may further comprise an expandable member such as a balloon and the balloon may be tapered.

In some embodiments, the outer surface of the first leg scaffold in the expanded configuration engages the outer surface of the expanded second leg scaffold thereby defining a mating region. The mating region may be disposed at least partially within the central passageway. The mating region may form a generally double D-shaped cross section.

The first leg and second leg scaffolds may traverse the aneurysm in a direction substantially parallel to one another or in some cases, they may cross each other. The downstream end of the first leg or second leg scaffold may be disposed downstream of the aneurysm or it may be disposed in an iliac artery. The downstream end of the docking scaffold may be disposed in a number of positions including upstream of the aneurysm, in the aneurismal sac, below the aneurysm or disposed in the blood vessel so as to traverse a renal artery bifurcation without inhibiting blood flow. The docking scaffold may comprise an expandable region that is adapted to linearly expand and contract in order to accommodate aneurysms of varying length. The docking scaffold may comprise a self-expanding region and a balloon expandable region as well as also including an external flange.

When the first double-walled filling structure is coupled with the first leg scaffold, the first double-walled filling structure at least partially fills the aneurysm when filled with the hardenable filling material. Some embodiments may further comprise a second double-walled filling structure having an outer wall and an inner wall, wherein the second filling structure is adapted to be filled with a hardenable fluid filling medium so that the outer wall conforms to an inside surface of the aneurysm and the inner wall forms a second substantially tubular lumen to provide a path for blood flow therethrough. The second double-walled filling structure may be coupled with the second leg scaffold in the expanded configuration. When the second double-walled filling structure is coupled with the second leg scaffold, the second double-walled filling structure at least partially fills the aneurysm when filled with the hardenable filling material. Some embodiments may also further comprise a third double-walled filling structure having an outer wall and an inner wall, wherein the third filling structure is adapted to be filled with a hardenable fluid filling medium so that the outer wall conforms to an inside surface of the aneurysm and the inner wall forms a third substantially tubular lumen to provide a path for blood flow therethrough. The third double-walled filling structure is disposed at least partially over the docking scaffold in the expanded configuration. When the third double-walled filling structure is coupled with the docking scaffold, the third double-walled filling structure often at least partially fills the aneurysm when filled with the hardenable filling material.

In some embodiments, the third double-walled filling structure is coupled with the docking scaffold and an upstream portion of the docking scaffold remains uncovered by the first double-walled filling structure in the expanded configuration. The uncovered upstream portion may be disposed upstream of the aneurysm. The uncovered upstream portion may also engage the blood vessel in the expanded configuration. When filled with filling medium, the third double-walled filling structure may seal an upper portion of the aneurysm thereby preventing blood flow between the outer wall of the third double-walled filling structure and an inner wall of the blood vessel. The third double-walled filling structure may be coupled with the docking scaffold and a downstream portion of the docking scaffold may remain uncovered by the third double-walled filling structure in the expanded configuration.

The docking scaffold may comprise a restraining element that limits expansion of at least a portion of the docking scaffold to a target diameter. The restraining element may be expandable. The restraining element may comprise a band that is disposed around the docking scaffold. Sometimes the restraining element may form a tapered region on one end of the docking scaffold in the expanded configuration.

In some embodiments, an upstream portion of the first leg scaffold remains uncovered in the expanded configuration and a downstream portion of the first leg scaffold may remain uncovered in the expanded configuration. The downstream portion of the first leg scaffold may be disposed in an iliac artery. The second leg scaffold may comprise an upstream portion that remains uncovered in the expanded configuration and a downstream portion of the second leg scaffold may also remain uncovered in the expanded configuration. The downstream portion of the second leg scaffold may be disposed in an iliac artery. The first and second leg scaffolds may be fixedly coupled together and either may comprise an external flange. Sometimes, the first or second leg scaffolds may comprise a self-expanding region and a balloon expandable region.

In still other embodiments, the first leg scaffold or second leg scaffold may comprise a sealing element disposed at least partially along the portion of the respective scaffold that is slidably received in the central passageway. The sealing element forms a seal between the outside surface of the first leg or second leg scaffold in the expanded configuration and the inside surface of the docking scaffold. The sealing element may be expandable and may have a chamfered surface.

In some embodiments, the system further comprise a third leg scaffold. The third leg scaffold is radially expandable from a contracted configuration to an expanded configuration. A portion of the third leg scaffold may be slidably received by the first or second leg scaffold such that a surface of the third leg scaffold in the expanded configuration engages a surface of the first or second leg scaffold. For example, the outside surface of the third leg scaffold may engage an inside surface of the first or second leg scaffold, or vice versa; the inside surface of the third leg scaffold may engage an outside surface of the first or second leg scaffold. An upstream end of the third leg scaffold may be disposed downstream of the aneurysm, for example in an iliac artery. Some embodiments may further comprise a fourth double-walled filling structure. The fourth filling structure has an outer wall and an inner wall and is adapted to be filled with a hardenable fluid filling medium so that the outer wall conforms to an inner surface of the aneurysm and the inner wall forms a fourth substantially tubular lumen to provide a path for blood flow therethrough. The fourth double-walled filling structure may be coupled with the third leg scaffold. When filled with the hardenable filling material, the fourth double-walled filling structure may at least partially fill an aneurysm in the iliac artery.

The system may also further comprise a fourth leg scaffold. The fourth leg scaffold is radially expandable from a contracted configuration to an expanded configuration. A portion of the fourth leg scaffold may be slidably received by the second leg scaffold such that a surface of the fourth leg scaffold in the expanded configuration engages a surface of the second leg scaffold. For example, the outside surface of the fourth leg scaffold may engage an inside surface of the second leg scaffold, or vice versa, the inside surface of the fourth leg scaffold may engage an outside surface of the second leg scaffold. An upstream end of the fourth leg scaffold may be disposed downstream of the aneurysm, for example in an iliac artery. Still some other embodiments may further comprise a fifth double-walled filling structure. The fifth filling structure has an outer wall and an inner wall. The fifth filling structure is adapted to be filled with a hardenable fluid filling medium so that the outer wall conforms to an inner surface of the aneurysm and the inner wall forms a fifth substantially tubular lumen to provide a path for blood flow therethrough. The fifth double-walled filling structure is coupled with the fourth leg scaffold. When filled with the hardenable filling material, the fourth double-walled filling structure at least partially fills an aneurysm in the iliac artery.

In some embodiments, the system may comprise a crown scaffold radially expandable from a contracted configuration to an expanded configuration. The crown scaffold has an upstream portion and a downstream portion. In the expanded configuration, the downstream portion is slidably received by the upstream end of the docking scaffold. The downstream portion may be slidably received in the central passageway such that an outside surface of the crown scaffold engages an inside surface of the docking scaffold. The upstream portion of the crown scaffold may engage a portion of the blood vessel upstream of the aneurysm. The crown scaffold may be self-expanding, balloon expandable or a combination thereof.

Sometimes, the docking scaffold comprises a divider disposed within the docking scaffold and adapted to separate the slidably received portion of the first leg scaffold from the slidably received portion of the second leg scaffold. The divider is often integrally formed with the docking scaffold. The divider may split the cross-section of the docking scaffold into two D-shaped cross-sections. The divider may be adapted to limit the length of the portion of the first leg scaffold and the portion of the second leg scaffold that are slidably received in the central passageway. Sometimes, the divider comprises an expandable structure, such as a double-walled filling structure, expandable from a contracted configuration to an expanded configuration. The expandable structure is configured to secure the slidably received portions of the first and second leg scaffolds when the expandable structure is expanded to the expanded configuration. This also helps form a seal to prevent blood flow past the expandable structure.

In some embodiments, the downstream end of the docking scaffold is bifurcated, for example, into a first portion and a second portion, wherein the first portion is adapted to slidably receive the first leg and the second portion is adapted to slideably receive the second leg. The docking scaffold may optionally be at least partially covered with a material.

In another aspect of the present invention, a method for treating an aneurysm in a blood vessel comprises advancing a docking scaffold through the blood vessel to a position upstream of the aneurysm and radially expanding the docking scaffold from a contracted configuration to an expanded configuration, wherein in the expanded configuration the docking scaffold engages a portion of the blood vessel upstream of the aneurysm. Advancing a first leg scaffold through the blood vessel toward the docking scaffold allows the first leg scaffold to be slidably received by the docking scaffold and radially expanding the first leg scaffold from a contracted configuration to an expanded configuration engages the first leg scaffold with at least a portion of an inner surface of the docking scaffold. Advancing a second leg scaffold through the blood vessel toward the docking scaffold allows the second leg scaffold to be slidably received by the docking scaffold and radially expanding the second leg scaffold from a contracted configuration to an expanded configuration engages the second leg scaffold with at least a portion of the inner surface of the docking scaffold. Advancing a first double-walled filling structure through the blood vessel moves the double-walled filling structure toward the aneurysm and filling the first double-walled filling structure with a fluid filling medium allows an outer wall of the first filling structure to conform to an inside surface of the aneurysm and an inner wall of the first filling structure forms a first substantially tubular lumen to provide a first blood flow path across the aneurysm. The first filling structure is coupled with at least one of the leg scaffolds in the expanded configuration.

Advancing the docking scaffold may comprise positioning at least a portion of the docking scaffold upstream of the aneurysm, across the aneurysm, downstream of the aneurysm or across a renal artery bifurcation without obstructing blood flow into the renal artery. The method may also comprise restraining a portion of the docking scaffold during radial expansion which may form a region of the docking scaffold having a constant predetermined diameter or a tapered region. Sometimes, restraining comprises limiting radial expansion of the docking scaffold with a band disposed circumferentially therearound.

Radially expanding the first leg and second leg scaffolds to the expanded configuration may comprise engaging the first leg scaffold with the second leg scaffold and advancing the first leg and second leg scaffolds may comprise crossing the first leg scaffold with the second leg scaffold.

The first filling structure may be disposed at least partially over the first leg scaffold in the expanded configuration. The method may also further comprise polymerizing the fluid filling medium in the first filling structure.

The method may further comprise advancing a second double-walled filling structure through the blood vessel toward the aneurysm. The method may also comprise filling the second double-walled filling structure with a fluid filling medium so that an outer wall of the second filling structure conforms to an inside surface of the aneurysm and an inner wall of the second filling structure forms a second substantially tubular lumen to provide a second blood flow path across the aneurysm. The second filling structure may be disposed at least partially over the second leg scaffold in the expanded configuration. The fluid filling medium may be polymerized in the second filling structure.

The method may also comprise advancing a third double-walled filling structure through the blood vessel toward the aneurysm and filling the third double-walled filling structure with a fluid filling medium so that an outer wall of the third filling structure conforms to an inside surface of the aneurysm and an inner wall of the third filling structure forms a third substantially tubular lumen to provide a third blood flow path across the aneurysm. The third filling structure may be disposed at least partially over the docking scaffold in the expanded configuration, and the method may comprise polymerizing the fluid filling medium in the third filling structure.

The method may also comprise polymerizing the fluid filling medium in the third filling structure. Filling the third double-walled filling structure may comprise sealing an upper portion of the aneurysm to prevent blood flow between an inner wall of the aneurysm and an outer wall of the third double walled filling structure. Radially expanding the docking scaffold comprises radially expanding an expandable member which may include inflating a balloon. In some embodiments, filling the first double-walled filling structure comprises filling the first filling structure while the balloon is inflated.

Sometimes, advancing the first or second leg scaffold may comprises positioning a portion of the scaffold in an iliac artery. Often, the method may further comprise sealing the first or second leg scaffolds within the docking scaffold to prevent blood flow between an outer surface of the first or second leg scaffolds and an inner surface of the docking scaffold. Sealing may include inflating a sealing element.

The method may also comprise advancing a third leg scaffold through the blood vessel toward the first or second leg scaffold and radially expanding the third leg scaffold. The third leg scaffold is advanced so that the third leg scaffold is slidably received by the first or second leg scaffold. The third leg scaffold is radially expanded from a contracted configuration to an expanded configuration. In the expanded configuration, the third leg scaffold engages at least a portion of a surface of the first or second leg scaffold, for example, the inside surface or the outside surface. Sometimes, a fourth double-walled filling structure with a fluid filling medium may also be advanced. The fourth filling structure is advanced so that an outer wall of the fourth filling structure conforms to an inside surface of the aneurysm and an inner wall of the fourth filling structure forms a fourth substantially tubular lumen to provide a fourth blood flow path. The fourth filling structure is disposed at least partially over the third leg scaffold in the expanded configuration. The fluid filling medium in the fourth filling structure may be polymerized. When the fluid filling medium is polymerized, the fourth filling structure may at least partially fill an aneurysm in the iliac artery.

Sometimes, a fourth leg scaffold is advanced through the blood vessel toward the second leg scaffold and radially expanded from a contracted configuration to an expanded configuration. The fourth leg scaffold is advanced so that the fourth leg scaffold is slidably received by the second leg scaffold. In the expanded configuration, the fourth leg scaffold engages at least a portion of the surface of the second leg scaffold, for example, the inside surface or the outside surface. A fifth double-walled filling structure with a fluid filling medium may be advanced. The fifth filling structure is advanced so that an outer wall of the fifth filling structure forms a fifth substantially tubular lumen to provide a fifth blood flow path. The fifth filling structure is disposed at least partially over the fourth leg scaffold in the expanded configuration. The fluid filling medium in the fifth filling structure may be polymerized. When the fluid filling medium is polymerized, the fifth filling structure may at least partially fill an aneurysm in the iliac artery.

The method may also comprise advancing a crown scaffold through the blood vessel to a position upstream of the aneurysm and radially expanding the crown scaffold from a contracted configuration to an expanded configuration. In the expanded configuration, the crown scaffold engages the upstream end of the docking scaffold. The crown scaffold may be slidably received in the central passageway such that an outside surface of the crown scaffold engages an inside surface of the docking scaffold. The upstream portion of the crown scaffold may engage a portion of the blood vessel upstream of the aneurysm.

These and other embodiments are described in further detail in the following description related to the appended drawing figures.

### BRIEF DESCRIPTION OF THE DRAWINGS

FIG. 1 illustrates the anatomy of an abdominal aortic aneurysm.

FIGS. 2A-2I show an exemplary method of treating an aneurysm with a docking station.

FIGS. 3A-3C illustrate how guidewires and scaffolds will often cross each other as they traverse the aneurysm.

FIG. 4A-4L illustrate another exemplary embodiment of a method for treating an aneurysm using double-walled filling structures and a docking station.

FIGS. 5A-5D show various configurations of a docking station scaffold relative to an abdominal aortic aneurysm.

FIGS. 6A-6C illustrate the use of a restraining element to control expansion of a scaffold.

FIGS. 7A-7C illustrate an embodiment of a sealing element.

FIGS. 8A-8D illustrate another embodiment of a sealing element.

FIG. 9 illustrates use of sealing elements.

FIG. 10 illustrates another use of sealing elements.

FIGS. 11A-11B illustrate yet another use of sealing elements.

FIGS. 12A-12C illustrate an inflatable sealing element.

FIG. 13 illustrates a configuration of scaffolds for treating aneurysms.

FIG. 14A-14B illustrate a configuration of a docking station scaffold with a crown scaffold relative to an abdominal aortic aneurysm.

FIGS. 15A-C illustrate configurations of a docking station scaffold with a divider element.

FIGS. 16A-C illustrate configurations of a docking station scaffold with a fillable divider element.

FIGS. 17A-B illustrate configurations of a docking station scaffold that is bifurcated.

FIG. 18 shows an embodiment of an iliac extension coupled with a docking scaffold.

FIGS. 19A-19C illustrate an embodiment of a variable length endograft.

FIG. 20 illustrates the use of a flexible docking scaffold in an aneurysm.

FIG. 21 illustrates the use of an external flange to help fix the endograft into position.

FIG. 22 shows a hybrid scaffold comprising a balloon expandable region and self-expanding region.

FIGS. 23A-23B illustrate various expandable members.

### DETAILED DESCRIPTION OF THE INVENTION

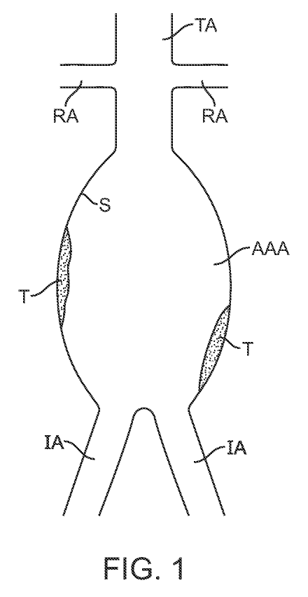

FIG. 1 illustrates the anatomy of an infrarenal abdominal aortic aneurysm comprising the thoracic aorta (TA) having renal arteries (RA) at an end above the iliac arteries (IA). The abdominal aortic aneurysm (AAA) typically forms between the renal arteries (RA) and the iliac arteries (IA) and may have regions of mural thrombus (T) over portions of its inner surface (S).

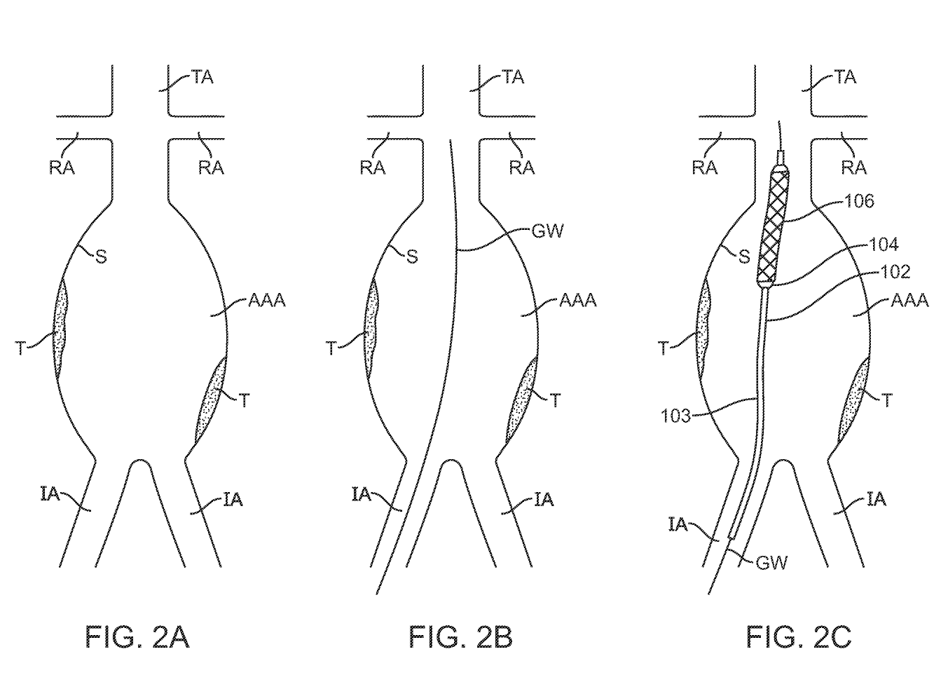

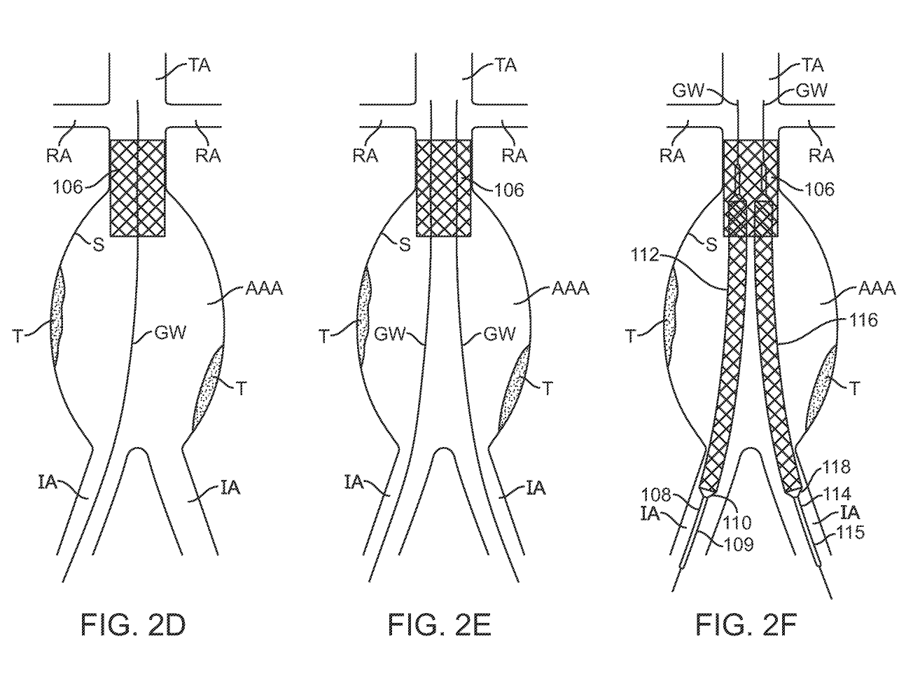

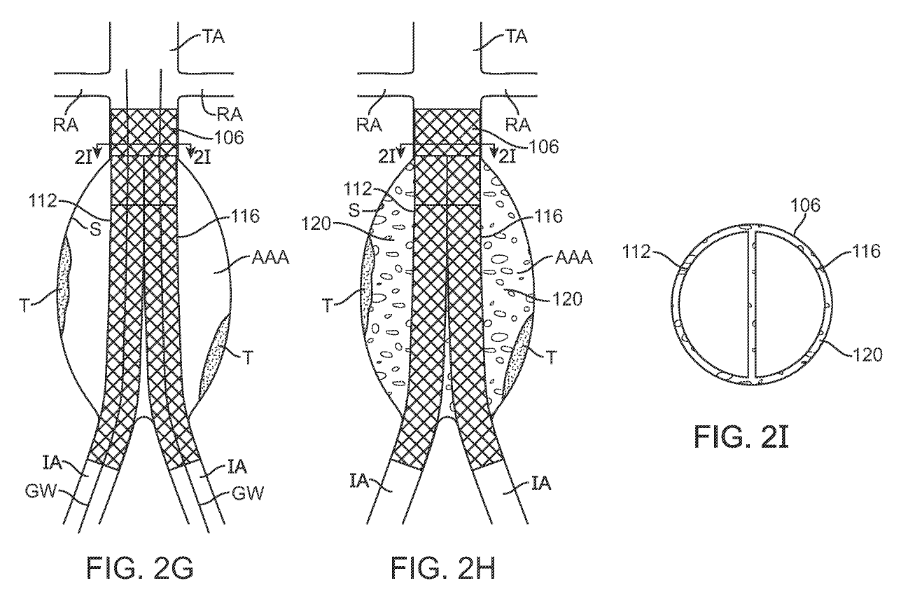

FIGS. 2A-2I show an exemplary method of treating an aneurysm using a docking station scaffold. FIG. 2A shows an infrarenal abdominal aortic aneurysm AAA similar to that in FIG. 1. In FIG. 2B, a guidewire GW is introduced using standard percutaneous or cutdown procedures into an iliac artery and the guidewire is advanced across the aneurysm toward the renal arteries RA. A docking station delivery system 102 is then advanced over the guidewire GW in FIG. 2C. The delivery system 102 includes a flexible catheter shaft 103 having a balloon 104 near its distal end and a docking station scaffold or scaffolding 106 positioned over the balloon 104. In some embodiments, the scaffolding 106 may be a bare metal stent-like scaffold, while in other embodiments the scaffolding 106 may be a covered stent-like scaffold. The covering may be a material such as Dacron™ or ePTFE, for example, materials that are commonly used in grafts and stent-grafts. An optional retractable outer sheath (not illustrated) may be positioned over the scaffolding 106 and balloon 104 in order to provide protection during delivery. The delivery catheter is advanced across the aneurysm so that approximately one-third of the docking station is disposed in the neck of the aneurysm with approximately two-thirds of the remaining scaffolding extending into the sac of the aneurysm AAA after expansion. One of ordinary skill in the art will appreciate that the position of the scaffold 106 may be adjusted in order to accommodate various anatomies.

In FIG. 2D, the balloon 104 is radially expanded so as to correspondingly expand scaffold 106 into engagement with the neck of the aneurysm. If scaffold 106 includes a covering (not illustrated), the covering material will also be expanded with the scaffold 106. In this embodiment, scaffolding 106 is a balloon expandable stent-like structure that may have numerous geometries such as disclosed in U.S. Pat. Nos. 4,733,665 to Palmaz, 5,733,303 to Israel et al. and 5,292,331 to Boneau. Many other geometries of stent-like structures are well reported in the patent and medical literature. In alternative embodiments, scaffolding 106 may also be a self-expanding stent-like structure, often fabricated from an alloy of nickel and titanium, such as Nitinol. After proper expansion and positioning of the scaffold 106 has been verified using fluoroscopy or other known techniques, the balloon 104 may be deflated and delivery catheter 102 removed from the patient, thus only expanded scaffold 106 and guidewire GW are left, as seen in FIG. 2D.

Referring now to FIG. 2E, a second guidewire GW is introduced using standard percutaneous or cutdown procedures from the contralateral leg, across the aneurysm AAA toward the renal arteries RA. In this exemplary embodiment, both guidewires are illustrated traversing the aneurysm AAA more or less parallel to one another, as seen in FIG. 2E. However, often the guidewires GW will cross and this will be discussed below. After both guidewires GW are properly positioned, a scaffolding delivery system 108 is advanced over the first guidewire GW, across the aneurysm AAA into the docking station 106. Delivery system 108 includes a catheter shaft 109 having a balloon 110 disposed near a distal end of the shaft 109 and a long scaffolding 112 disposed over the balloon 110. Scaffolding 112 may also optionally be covered with a material such as Dacron™ or ePTFE, as described above with respect to docking station 106, or it may be a bare metal or polymer scaffold. An optional outer sheath (not illustrated) may also be used to protect and/or constrain the balloon 110 and scaffolding 112 during delivery. The scaffolding 112 is balloon expandable although it may also be self-expanding and generally takes the same form as the docking station 106 with the major difference being its length. Scaffolding 112 is long enough the traverse the aneurysm AAA while still providing long enough proximal and distal ends that can expand into and engage the docking station 106 and the iliac arteries. Scaffolding 112 is advanced into the docking station 106 approximately one-third of the way, although clearly this may be modified as required.

FIG. 2F also shows another scaffolding delivery system 114 advanced over the second guidewire GW. Delivery system 114 is similar to delivery system 108 and includes a catheter shaft 115 having a balloon 118 disposed near the distal end of shaft 115 and scaffolding 116 is disposed over the balloon 118. Scaffolding 116 may also be covered with a material similar to that described above with respect to scaffolding 112 or it may remain uncovered. An optional outer sheath (not illustrated) may also be used to protect and/or constrain the balloon 118 and scaffolding 116 during delivery. Scaffolding 116 is balloon expandable, but may be self-expanding and generally takes the same form as scaffolding 112. Scaffolding 116 is advanced into docking station 106 approximately one-third of the way, although this may be adjusted as required. FIG. 2F shows both scaffolds 112, 116 traversing the aneurysm AAA parallel to one another, yet as previously discussed, often guidewires GW will cross, thus scaffolds 112 and 116 would also cross as they traverse the aneurysm.

Referring now to FIG. 2G, once both scaffolds 112, 116 have been positioned across the aneurysm and into docking station 106, balloons 110, 118 are inflated so as to radially expand scaffolds 112, 116 such that one end of each scaffold engages an iliac artery while the opposite end of each scaffold engages at least a portion of the inner surface of docking station 106. If the scaffolds 112, 116 are covered, the covering material (not illustrated) will also expand with the scaffold. Each balloon 110, 118 may be inflated independently of one another, or in preferred embodiments, both balloons 110, 118 are inflated simultaneously, thereby also expanding both scaffolds 112, 116 simultaneously. This helps to ensure that both scaffolds expand symmetrically with respect to one another and against one another so that the ends of each scaffold expand into the preferred double D-shaped configuration within the docking station 106, as seen in FIG. 2I. Other geometries of the mating ends of scaffolds 112 and 116 are possible, such as circular, elliptical, etc. and ideally the region where the two scaffolds meet should have minimal impact on disrupting blood flow thereacross. Balloons 110 and 118 are then deflated and delivery catheters 108 and 114 are removed from the treatment site.

The docking station 106 and two scaffold legs 112, 116 now form the basis of a blood pathway that will exclude aneurysm AAA. In the embodiment where scaffolds 112, 116 include a covering material such as Dacron™ or ePTFE, the lumens are fully formed and blood will flow from the thoracic aorta TA into docking station 106 and then flow is bifurcated across aneurysm AAA into both iliac arteries IA. In the embodiment where the scaffolds 112, 116 do not have a covering material and are bare metal or bare material scaffolds, blood can still flow through the sidewall apertures of the expanded scaffolds 112, 116. Thus, as shown in FIG. 2H, a filling material 120 may be used to fill the aneurismal sac so that blood flow remains within the lumens created by scaffolds 112, 116. An intravascular catheter (not illustrated) may be advanced into one or both expanded scaffolds 112, 116 and either placed against an aperture in one of the scaffold sidewalls, or the catheter may be advanced through one of the sidewall apertures. A hardenable filling material 120 may then be delivered to fill the aneurismal space. The filling material 120 may be viscous enough or its size may be large enough to prevent backflow into the scaffold 112, 116 or a balloon catheter may be expanded within the scaffolds to prevent backflow. Once the filling material 120 has hardened, a bifurcated lumen for blood flow across the aneurysm is formed. Furthermore, the hardening material may help lock the scaffolds in position relative to the aneurysm thereby preventing future migration. FIG. 2I shows a cross section of the scaffolds taken across line 2I-2I in FIG. 2H. The docking station 106 will generally take a round shape while the two iliac scaffolds 112, 116 will preferably form opposed double D-shapes. Filling material 120 will fill any gaps between the stents and aneurismal wall. Further information on using a hardening material to fill an aneurysm around scaffolding structures may be found in U.S. patent application Ser. No. 11/444,603 (Attorney Docket No. 025925-001810US), the entire contents of which are fully incorporated herein by reference.

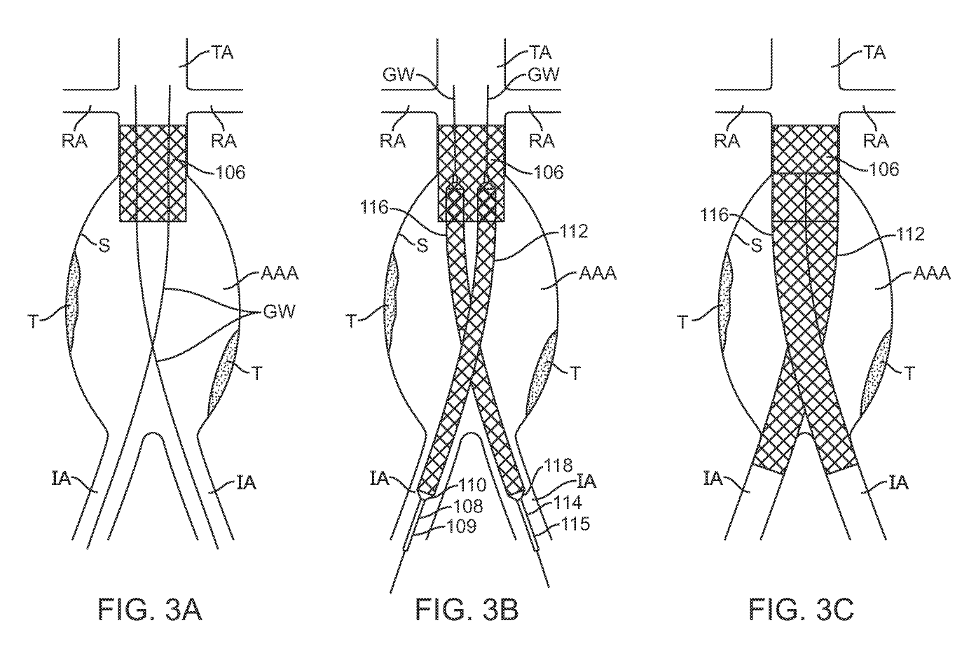

As previously mentioned, FIGS. 2A-2I show both guidewires GW and scaffolds 112, 116 traversing the aneurysm AAA in a generally parallel fashion. However, often times, due to the bias of the guidewires, the guidewires GW will cross each other as they traverse the aneurysm AAA, as seen in FIG. 3A. Thus, as scaffolds 112, 116 are advanced over the guidewires GW across the aneurysm AAA, they too will cross, as seen in FIG. 3B. FIG. 3C shows how both scaffolds 112, 116 will cross each other in the expanded configuration as well.

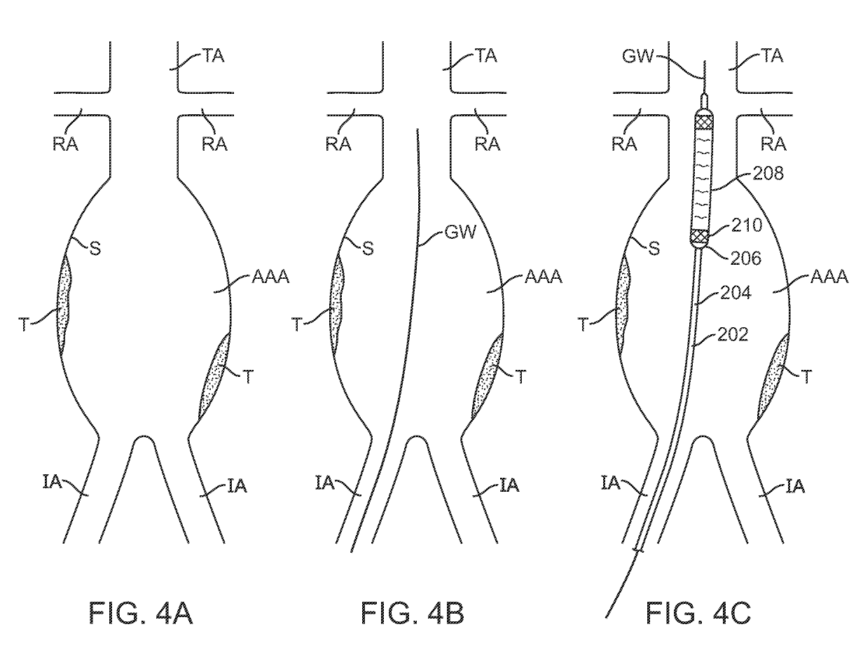

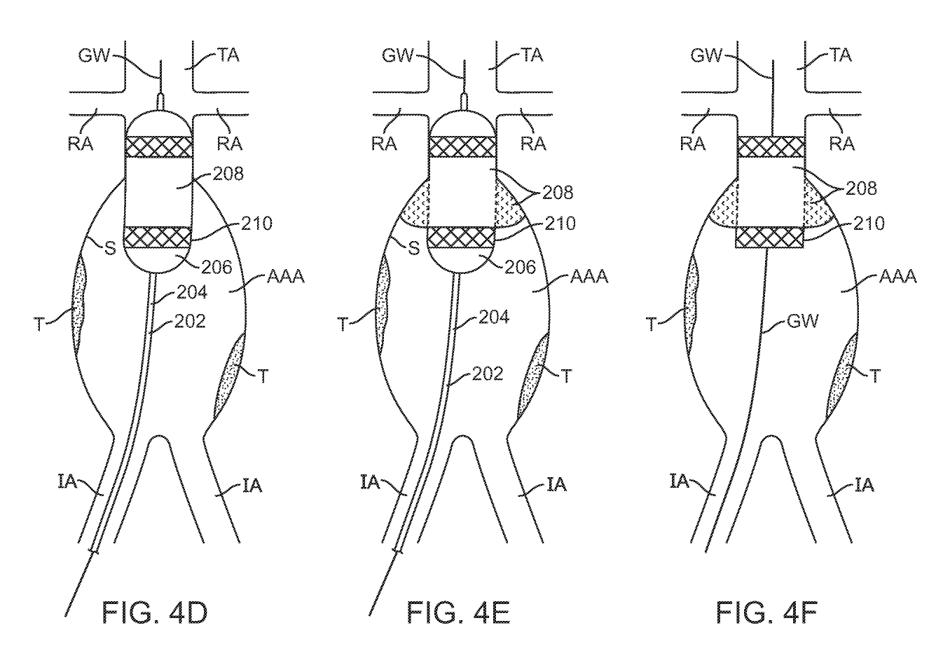

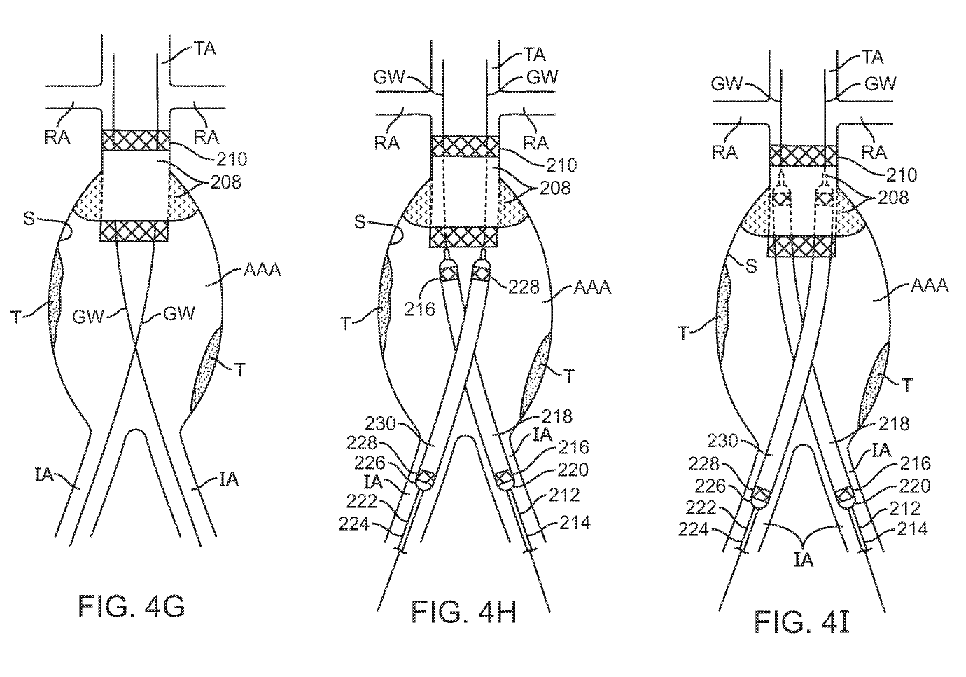

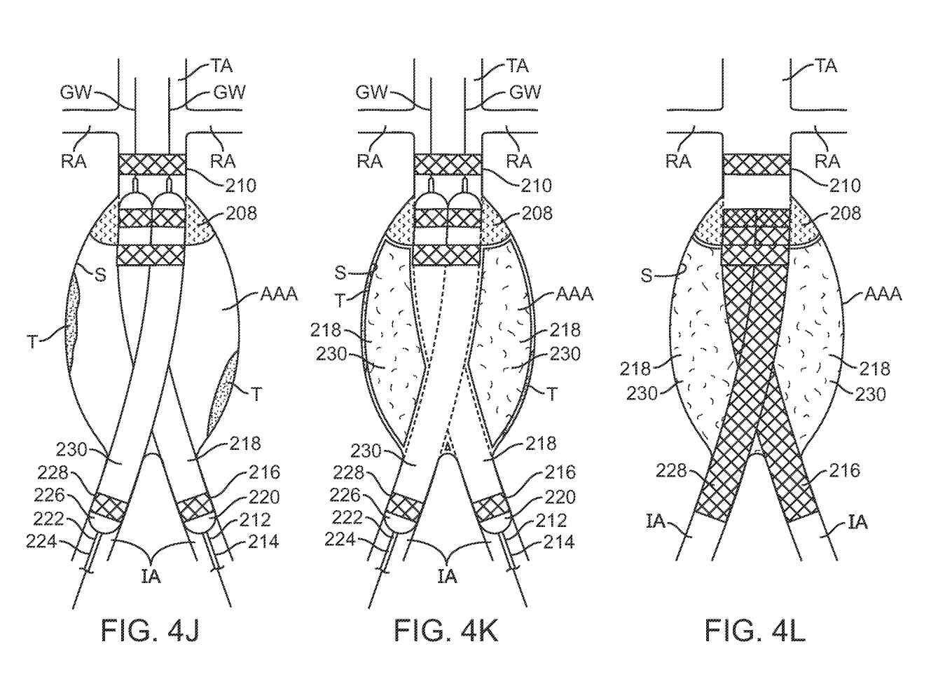

A preferred embodiment for treating an abdominal aortic aneurysm is illustrated in FIGS. 4A-4L. The major difference between this embodiment and the previous embodiment of FIGS. 2A-2I is the use of double-walled filling structures to help anchor the scaffolds in position and to seal the aneurismal sac, as will be described below.

Referring now to FIG. 4A, an abdominal aortic aneurysm AAA is located below the thoracic aortic TA, between the renal arteries RA and the iliac arteries IA. Sometimes, the aneurysm AAA may have mural thrombus T on an inner surface S of the aneurysm AAA. In FIG. 4B, a guidewire GW is introduced using standard percutaneous or cutdown procedures through an iliac artery, across the aneurysm AAA and toward the renal arteries RA. An endograft delivery system 202 is then advanced over the guidewire GW towards the renal arteries RA in FIG. 4C. Delivery system 202 includes a catheter shaft 204 having a balloon 206 near its distal end. A radially expandable scaffolding 210 is positioned over the balloon 206 and a double-walled filling structure 208 is disposed over the scaffolding 210. The filling structure 208 covers most of scaffolding 210, but in preferred embodiments scaffolding 210 has a region on both ends that is not covered by filling structure 208. The scaffolding 210 is a stent-like support structure, similar to those discussed with respect to FIGS. 2A-2I above. The double-walled filling structure is an ePTFE sealed bag coated on the inside with polyurethane that is wrapped around scaffold 210 so that it may be filled with a hardenable filling material to help seal the scaffolding around the aneurysm and create a lumen for blood flow. Further details on the double-walled filling structure are disclosed in U.S. Patent Publication No. 2006/0212112 (Attorney Docket No. 025925-001610US), the entire contents of which are fully incorporated herein by reference.

In FIG. 4D, balloon 206 is radially expanded, often by inflating the balloon 206 with saline and/or contrast media and this correspondingly expands the filling structure 208 and scaffold 210 such that the filling structure 208 and the scaffold 206 engage a wall of the blood vessel above the aneurysm AAA. In this embodiment, an exposed, uncovered region of scaffold 210 will expand directly into engagement with the blood vessel wall and a portion of filling structure 208 will also directly engage the blood vessel wall. In preferred embodiments, approximately one-third of the scaffold 210 will be positioned above the aneurysm AAA and approximately two-thirds of the scaffold 210 will be positioned in the aneurismal sac, although one will appreciate that different positions are possible depending on physician preference and patient anatomy. Additionally, in other embodiments, more or less of scaffold 210 may be covered by the filling structure 208.

In FIG. 4E, filling structure 208 is filled with a hardenable filling material such as PEG or another polymer that may be polymerized in situ. In FIG. 4E, the filling structure 208 is filled via a filling tube (not shown) that may run along side the delivery catheter shaft 204 or via a lumen in the delivery catheter shaft 204. The filling tube is discussed in greater detail in U.S. patent application Ser. No. 12/429,474 (Attorney Docket No. 025925-002610US), the entire contents of which are incorporated herein by reference. Additionally, the filling structure 208 is filled preferably while balloon 206 is still inflated. This helps to maintain a lumen for blood flow and also helps to prevent collapsing of the scaffold 210 as the filling structure 208 is filled. In some embodiments, the filling structure 208 may be filled after the balloon 206 has been deflated. In either case, it may be desirable to monitor pressure of the filling material as it fills the filling structure 206 and/or the volume of filling material introduced into the filling structure 208. Additional information on pressure and volume monitoring of filling structures is disclosed in U.S. patent application Ser. No. 12/429,474 (Attorney Docket No. 025925-002610US), previously incorporated by reference. Filling status may also be monitored by observing the filling structure 208 under fluoroscopy or ultrasound as it is filled. FIG. 4E shows the filling structure 208 filled while balloon 206 is still expanded. Once filled, filling structure 208 partially fills the aneurismal sac and seals off the top portion of aneurysm AAA from blood flow. A lumen is therefore created for blood flow through the inside of scaffold 210, which is also further anchored into position not only by the expanded scaffold 210 but also by the filled filling structure 208. After the filling structure has been filled and hardened, delivery catheter 204 is removed, leaving only the scaffold 210, filled filling structure 208 and guidewire GW in place, as seen in FIG. 4F. In some embodiments, a pre-filling of filling structure 208 may be used prior to filling with the hardenable material. This is performed to help unfurl the filling structure 208 and pre-filling the filling structure 208 with a fluid such as carbon dioxide, saline or contrast media helps the operator estimate the volume of hardenable filling material to be used during the final filling of the filling structure 208.

Once docking scaffold 210 is expanded into position, it will serve as a docking station for two additional endografts which will form the legs of the system and provide lumens for blood flow across the aneurysm AAA into the iliac arteries IA. In FIG. 4G, a second guidewire GW is percutaneously introduced and advanced from the contralateral limb across the aneurysm AAA, through the scaffold 210 upstream toward the renal arteries. In FIG. 4G, the guidewires GW are shown crossing each other which often occurs, although as previously indicated above, the guidewires may also traverse the aneurysm in a generally parallel fashion. In FIG. 4H, two additional endograft systems are advanced over the guidewires GW. A first endograft delivery system 212 comprises a catheter shaft 214 having a balloon 220 coupled to the shaft 214 near the distal end. A scaffold 216 is positioned over the balloon 220 and a filling structure 218 is positioned over most of the scaffold 216 while still leaving the ends of scaffold 216 exposed. The scaffold 216 and filling structure 218 generally take the same form as scaffolding 210 and filling structure 208 described above, with the major differences being their lengths and diameters. A second endograft delivery system 222 also comprises a catheter shaft 224 having a balloon 226 coupled to the shaft 224 near the distal end. Also, a scaffold 228 is positioned over the balloon 226 and a filling structure 230 is positioned over most of the scaffold 228 while still leaving the ends of scaffold 228 exposed. The scaffold 228 and filling structure 230 generally take the same form as scaffolding 216 and filling structure 218.

In FIG. 4I, both endograft delivery systems 212, 222 are advanced such that the docking scaffold 210 with filled filling structure 208 slidably receives an end of both scaffolds 216, 228 and optionally a portion of both filling structures 218, 230. In this embodiment, the scaffolds 216, 228 are advanced approximately one-third of the way into the docking scaffold 210 although one of skill in the art will appreciate that this distance may be adjusted as required in order to accommodate different anatomies.

In FIG. 4J, both balloons 220, 226 are inflated thereby expanding both scaffolds 216, 228 along with their respective filling structure 218, 230. The balloons 220, 226 in this embodiment are inflated simultaneously in order to help ensure symmetric expansion of both scaffolds 216, 228 and both filling structures 218, 230. However, in some embodiments, inflation may be sequentially performed. The balloons 220, 226 are expanded so as to ensure that one end of each scaffold expands into engagement with the docking scaffold 210 while the other end of each scaffold expands into engagement with an iliac artery IA. In this embodiment, the scaffolds 216, 228 are balloon expandable, however, they may also be self-expanding.

After expansion of the balloons 220, 226 the filling structures are filled with a hardenable filling material such as PEG which can be polymerized in situ. This is seen in FIG. 4K. As discussed above, in some embodiments, prior to filling the filling structures 218, 230 with the hardenable filling material, they may be pre-filled with carbon dioxide, contrast media, saline or a combination thereof in order to help unfurl each filling structure and also to give a preliminary indication of volume and/or pressure to use to fill the structures. Also, in this embodiment, the filling structures 218, 230 are filled while balloons 220, 226 are inflated in order to help prevent crushing of the underlying scaffolds 216, 228 although in other embodiments, the balloons need not be inflated during this step. FIG. 4L illustrates the final configuration of the endograft system after the delivery catheters and guidewires have been removed from the patient. A docking scaffold 210 is upstream of the aneurysm AAA and two scaffolds 216, 228 are expanded with one end in the docking scaffold 210 and the other end in the iliac arteries IA. Each scaffold 210, 216 and 228 has a filling structure 208, 218, 230 which is filled with a hardenable material to help anchor each scaffold in position and to help seal the aneurismal sac off from blood flow thereby forcing blood to flow through the lumens created by the scaffolds and their respective filling structures. While this embodiment shows one filling structure associated with each scaffold, in other embodiments some scaffolds may have a corresponding filling structure while others will not.

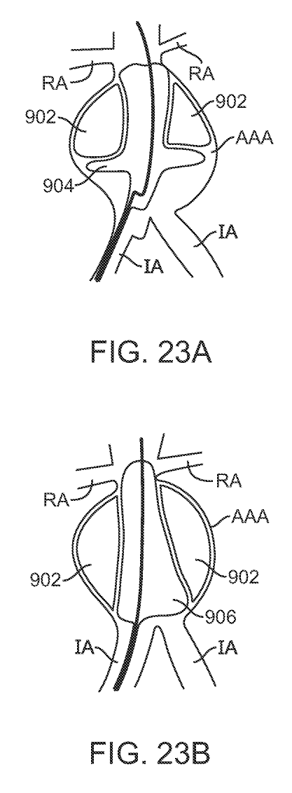

The balloons used to deploy the scaffolds and filling structures are often similar to balloons used for angioplasty and stenting. However, in some cases, it may be helpful to use alternatively shaped balloons to help ensure proper deployment of the filling structures. For example, in FIG. 23A, a balloon 904 having a lower flange region may be used to help ensure that expansion of the filling structures 902 is limited to a defined region. Or, for example, in FIG. 23B, a tapered balloon 906 is used to shape the filling structures 902 so that an internal chamfer is formed, thereby helping to ensure a smooth transition for receipt of the iliac extension legs.

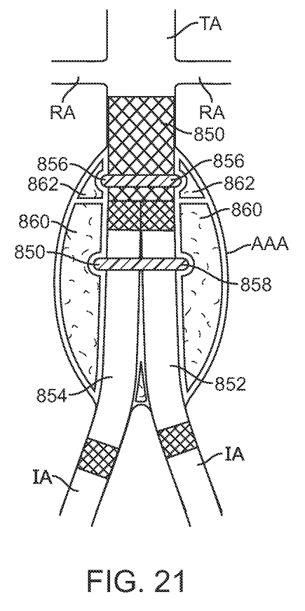

Now referring to FIG. 21, an optional external flange on the docking scaffold and/or the iliac leg scaffolds may further secure each scaffold into position. In FIG. 21, the docking scaffold 850 includes an outer annular ring or flange 856. This flange may be fabricated from a metal or polymer and it expands with the scaffold during deployment. Because it has a larger profile than the scaffold body, the filling structure 862 will expand around it and once the filling medium has hardened, the flange will be locked into position. Similarly, an optional flange 858 may be included in one or both of the iliac leg scaffolds 852, 854 to provide an area for filling structures 860 to expand around and capture.

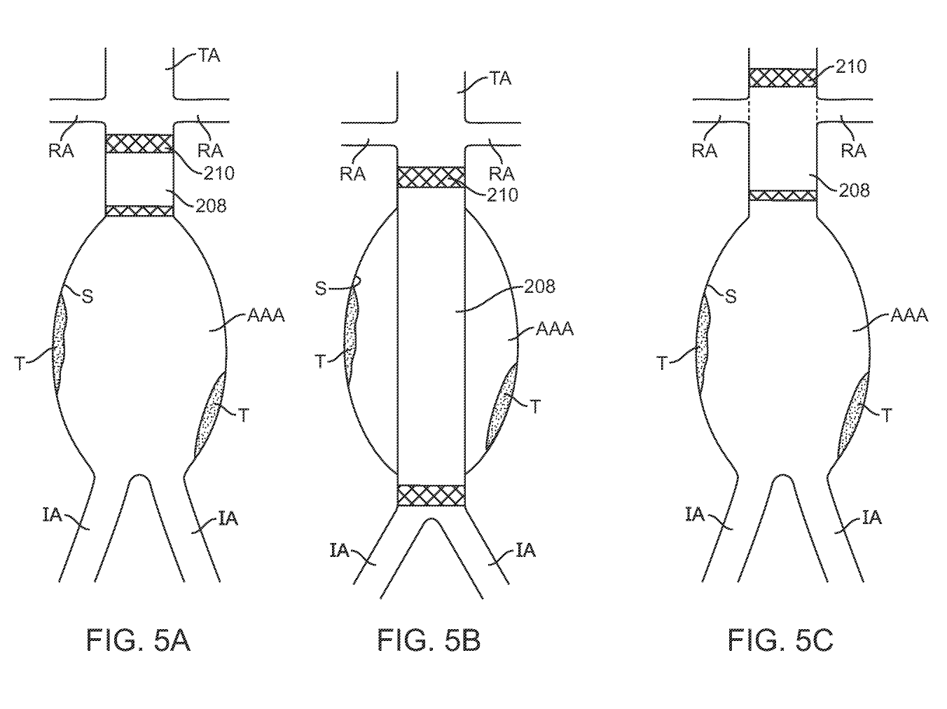

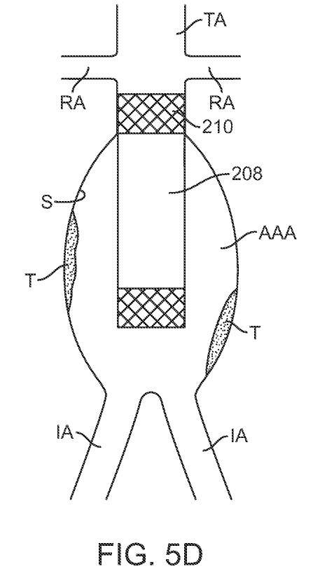

In the embodiment discussed above with respect to FIGS. 4A-4I, the filling structure is shown disposed over the scaffold. Other configurations are possible. For example, the scaffold may be disposed axially separated from the filling structure in order to reduce overall delivery profile. Additional disclosure on delivery system configurations may be found in U.S. patent application Ser. No. 12/429,474 (Attorney Docket No. 025925-002610US), previously incorporate herein by reference. Additionally, the docking scaffold 210 is shown positioned with approximately one-third of its length positioned in the aorta upstream of the aneurysm while the remainder of the scaffold is positioned in the aneurismal sac. One of ordinary skill in the art will appreciate that different configurations of the docking scaffold 210 may be utilized. For example, FIG. 5A shows a docking scaffold 210 with optional filling structure 208 positioned in the aorta upstream of the aneurysm and below the renal arteries RA. FIG. 5B shows yet another variation where the docking scaffold 208 is positioned with an upper portion in the aorta upstream of the aneurysm, a main section traverses the aneurysm and a lower portion is positioned below the aneurysm just before iliac bifurcation. FIG. 5C shows still another variation where the docking scaffold 210 is placed in the aorta above the aneurysm and across the renal arteries RA. In this embodiment, the scaffold 210 and optional filling structure 208 have windows or lateral apertures that permit blood flow from the aorta to the renal arteries without significantly obstructing flow. FIG. 5D illustrates yet another variation where the docking scaffold 210 is placed partially in the aorta above the aneurysm and a downstream portion is in the aneurismal sac. Any of the embodiments shown in FIGS. 5A-5D may also optionally include a filling structure 208 which generally takes the same form as filling structures previously described.

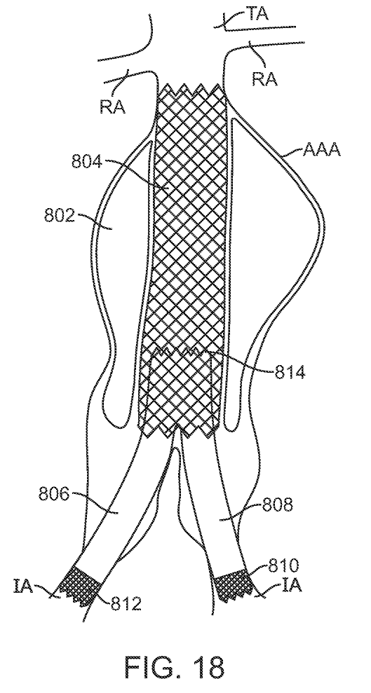

Any of the docking scaffolds may be coupled with two iliac leg extensions as described herein. Most of the embodiments disclosed use two discrete iliac leg extensions delivered separately from both iliac arteries. However, in some embodiments, the iliac leg extensions may be of integral construction rather than discrete. For example, in FIG. 18, a docking scaffold 804 having a filling structure 802 is disposed across the aneurysm AAA such that one end is upstream of the aneurysm and the opposite end is downstream of the aneurysm. An iliac leg extension of unitary construction having two iliac legs 806, 808 coupled together is then slidably received and radially expanded in the downstream portion of the docking scaffold 804 such that blood flow is bifurcated to each iliac artery. The iliac leg extension may be a stent-like scaffold only, it may be a covered graft or it may be a graft with scaffolds only at its ends such as the embodiment in FIG. 18 which has scaffolds 814, 812 and 810 at its ends. One or more optional filling structures may also be coupled with the iliac extension.

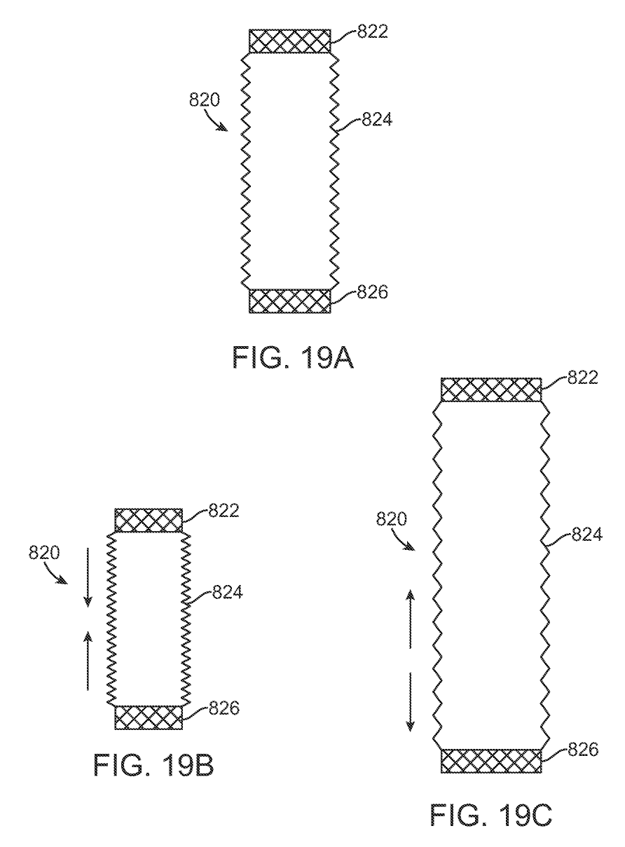

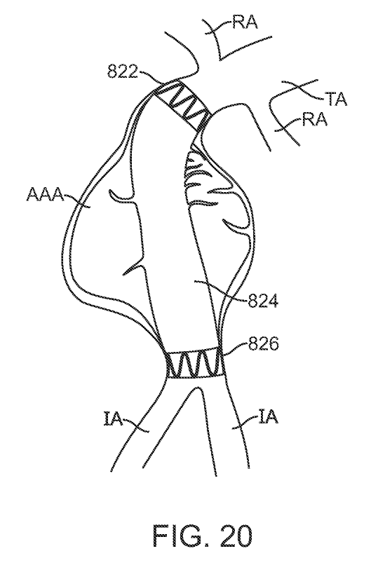

Often the docking scaffold is a fixed length. While some foreshortening may occur during radial expansion, the docking scaffold generally does not change length significantly. This requires the physician to accurately determine the required length prior to deployment and also requires a number of different length to be inventoried. An accordion-like docking scaffold allows a single scaffold to accommodate a number of aneurysm lengths. FIGS. 19A-19C illustrate an exemplary embodiment of a variable length docking scaffold. In FIG. 19A, the docking scaffold 820 includes an accordion-like main body 824 and stent-like ends 822, 826. The main body 824 may be a graft alone or it may also be supported by a scaffold structure such as a stent. The graft material may be Dacron woven to allow axial extension and compression or it may be ePTFE which will also stretch and compress depending on the material properties such as internodal distance. Other materials may also be used. Both ends, 822, 826 may include balloon expandable or self-expanding stents to help anchor the docking scaffold in position. FIG. 19B shows the docking scaffold in a compression configuration so that it may accommodate a shorter aneurysm and FIG. 19C shows the docking scaffold in an elongated configuration for a longer aneurysm. In addition to providing a scaffolding that can accommodate varying lengths, this embodiment is also more flexible and thus may accommodate bends and other tortuosity often seen in aneurysms, such as in FIG. 20. While this embodiment is described with respect to the docking scaffold, one of skill in the art will appreciate that this embodiment may also be used in the iliac legs or other portions of the system.

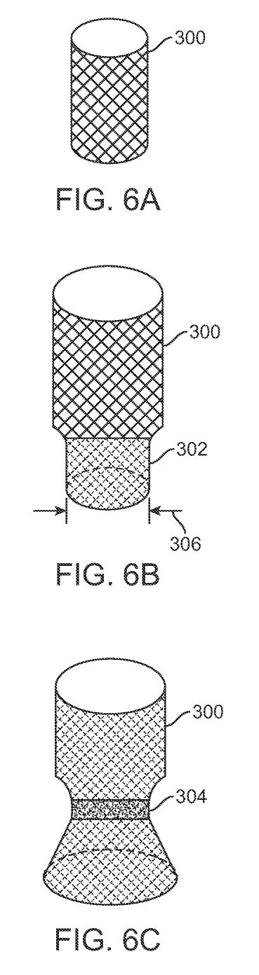

FIGS. 6A-6C illustrate another feature of the docking scaffold which may optionally be included in any of the embodiments disclosed herein. FIG. 6A illustrates the standard docking scaffold 300 which is generally cylindrically shaped with a constant diameter. In some cases, it may be desirable to expand the docking scaffold 300 so that a lower end expands to a constant diameter every time. This standardizes the docking region of scaffold 300 and allows more consistency in mating the docking scaffold with the two legs. Additionally, this allows the upper portion of the scaffold to accommodate a variety of vessel anatomies and sizes without interfering with the docking aspect of the scaffold. FIG. 6B illustrates an exemplary embodiment of a docking scaffold 300 having a restraining member 302 disposed over a lower portion of the scaffold 300. The restraining member 302 may be a corset like band of material that limits expansion of the scaffold, or the scaffold itself may have shorter struts that expand less than other regions of the scaffold. The restraining member 302 or shorter struts allow the lower portion of scaffold 300 to expand to a predetermined diameter 306 which is sized so as to mate with the two endograft legs. In still other embodiments, a restraining member 304 or the scaffold design itself may be used to limit expansion of the docking scaffold to create a tapered or flared region such as seen in FIG. 6C. The tapered or flared region may be used to help guide the endograft legs into the docking scaffold 300 during assembly of the endograft system in situ.

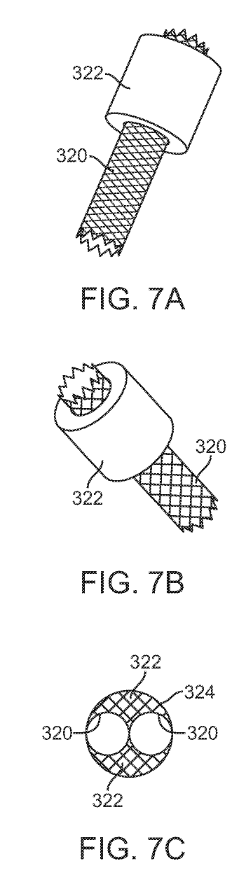

FIGS. 7A-7C illustrate still another feature of the docking scaffold system which may optionally be included in any of the embodiments disclosed herein. In order to help ensure sealing between the docking scaffold and the two legs, a sealing element may be disposed around one or both of the leg scaffolds. The sealing element may be used to fill gaps as well as cause thrombus formation. FIG. 7A illustrates a scaffold 320 having such a sealing element 322. FIG. 7B is a perspective view showing the sealing element. The sealing element 322 may be a foam-like plug or a spongy, material that can be compressed to minimize profile during delivery. Exemplary materials for the sealing elements may include, but are not limited to polyurethane, polycarbonate, polyester, ePTFE, polyolefins, parylene, gelatin, silicone and the like. A sheath may be used to constrain the sealing element 322 during delivery. Upon retraction of the sheath the sealing element expands to fill any gaps. In addition to sealing any gaps, the sealing element may be fabricated from a material or contain a therapeutic agent which causes thrombosis thereby providing additional sealing ability. FIG. 7C shows an exemplary cross-sectional view of the docking scaffold 324 having two leg scaffolds 320 expanded and engaged therein. Sealing elements 322 on both leg scaffolds 320 fill the gaps between the docking scaffold 324 and the two leg scaffolds 320 to prevent blood flow therethrough.

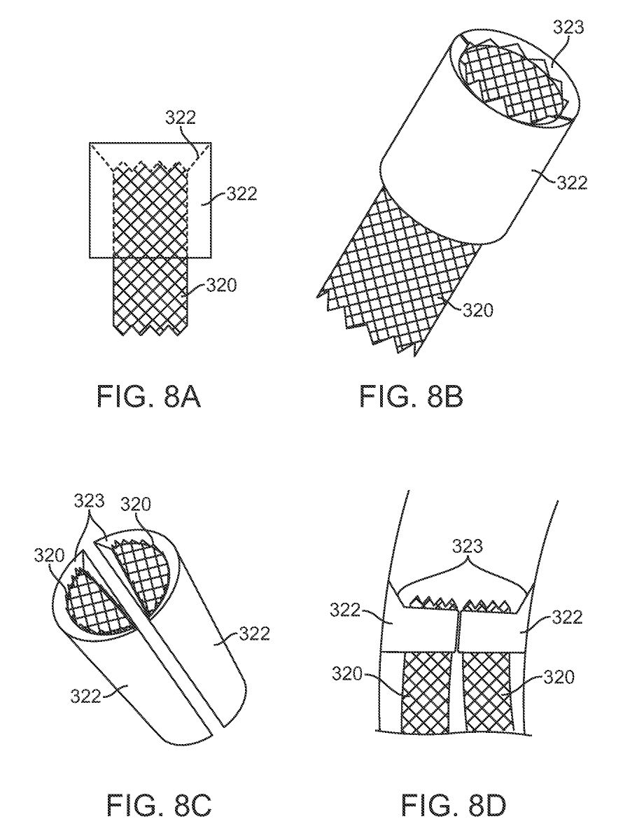

Shaped sealing elements may also facilitate blood or fluid flow across a sealed region. For example, FIG. 8A illustrates a side view of a scaffold 320 having a sealing element 322 disposed on one end. An internal chamfer 323 provides a smoother transition for fluids to enter the scaffold 320. FIG. 8B illustrates a perspective view of FIG. 8A. FIG. 8C shows a perspective view of an exemplary embodiment where two sealing elements 322 are disposed against one another, thereby forming a double D-shaped region. Again, the chamfer 323 provides a smooth transition. FIG. 8D shows a side view of FIG. 8C.

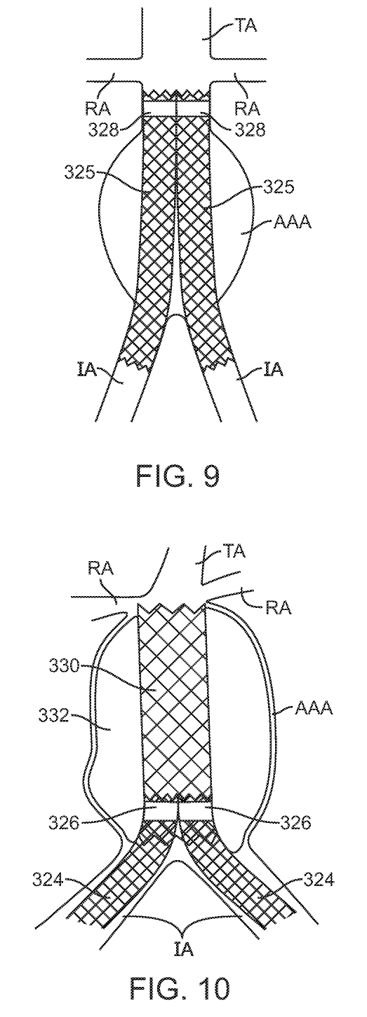

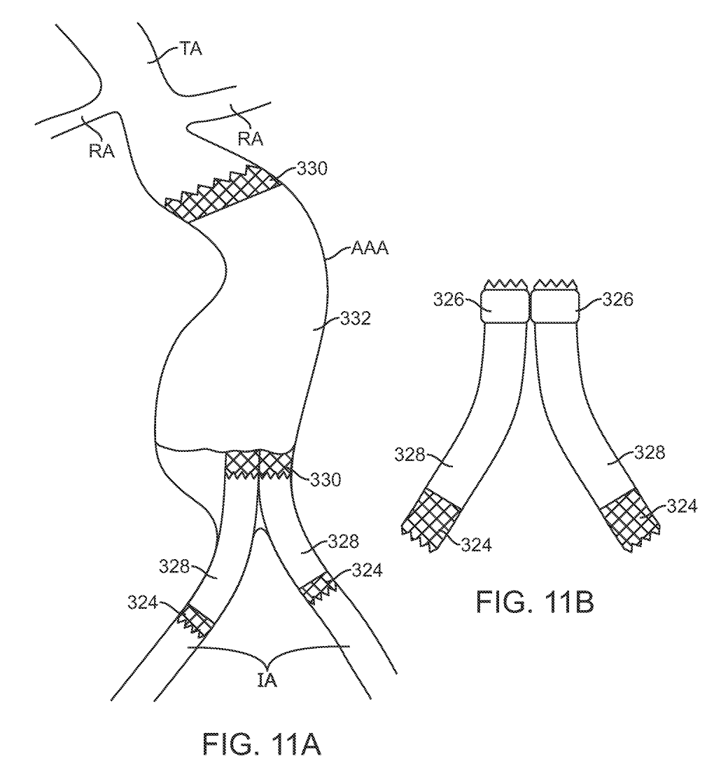

Additionally, FIGS. 9 and 10 illustrate how the sealing elements may be used in alternative embodiments. For example, in FIG. 9 two scaffolds 325 are placed side-by-side in an aneurysm AAA. An upper portion of each scaffold 325 is positioned upstream of the aneurysm AAA and sealing elements 328 form a seal between the scaffolds 328 and blood vessel wall. Both scaffolds 325 traverse the aneurysm AAA and an opposite end of each scaffold 325 is positioned in an iliac artery IA. In the embodiment of FIG. 9, the scaffolds 325 are preferably covered with a cover such as ePTFE or Dacron so that blood flow follows the lumen created by the scaffolds 325 into the iliac arteries, IA, thereby excluding the aneurysm AAA. FIG. 10 illustrates another embodiment where the sealing elements 326 are used to form a seal. In FIG. 10, a docking scaffold 330 with double-walled filling structure 332 is positioned with an upper portion in the neck of the aneurysm AAA and the main body traversing the aneurysm AAA. Iliac leg scaffolds 324 dock with the docking scaffold 330 and sealing elements 326 seal the system to ensure blood flow only through the endograft lumens. In the embodiment of FIG. 10, the docking scaffold 330 may optionally be covered along with the iliac leg scaffolds 324 with a cover such as ePTFE or Dacron 328. FIGS. 11A-11B illustrate such an embodiment. In FIG. 11A, a docking scaffold 330 is positioned partially upstream of the aneurysm AAA and a filled filling structure 332 partially fills the aneurismal space. Two iliac scaffolds 324 dock with docking scaffold 330 and their opposite ends are positioned in the two iliac arteries IA. Sealing elements 326 on the upstream portion of scaffolds 324 help form a seal and a covering material such as ePTFE or Dacron cover the iliac scaffolds 328 to restrict blood flow to the lumen created by the iliac scaffolds 324. FIG. 11B shows the two iliac scaffolds 324 adjacent one another and having sealing elements 326 at one end, a covered middle portion and an uncovered scaffold portion on the opposite end.

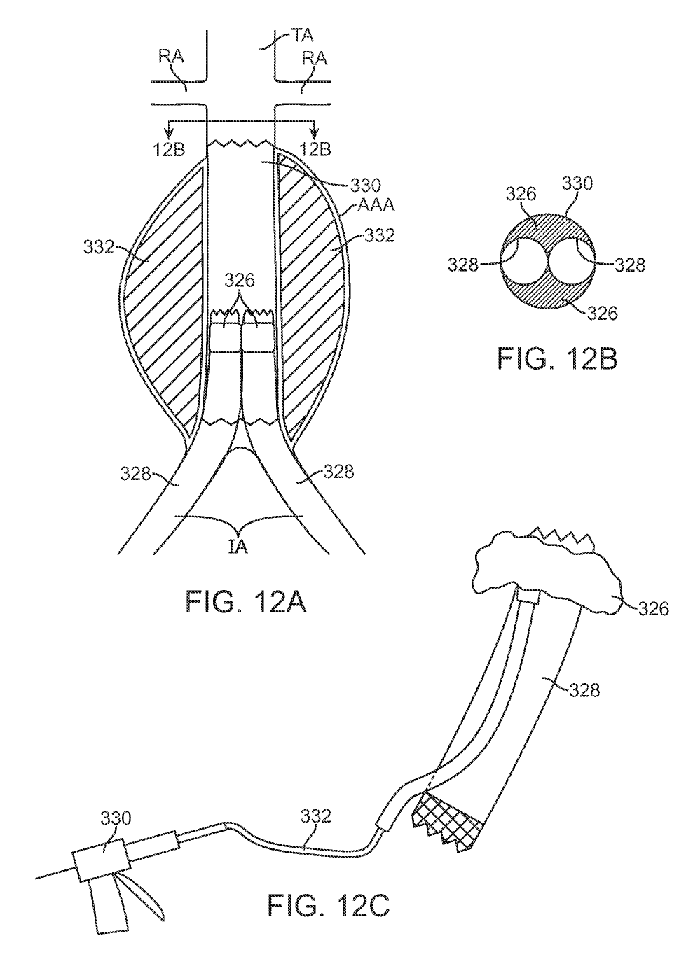

In still other embodiments, the sealing elements may be expandable or inflatable members. FIGS. 12A-12C illustrate an exemplary embodiment. In FIG. 12A, a docking scaffold 330 is placed in the vessel and partially across the aneurysm AAA. A filling structure 332 is filled with hardenable filling material such as PEG and iliac scaffold legs 328 are docked into the docking scaffold 330. The iliac scaffold legs 328 may be grafts alone or they may be supported by a stent-like scaffold structure. Expandable sealing elements 326 on each iliac scaffold leg 328 form a seal. FIG. 12B shows a cross section along the line 12B-12B in FIG. 12A and shows how the expandable sealing elements 326 fill the gaps between the docking scaffold 330 and the two iliac scaffold legs 328. FIG. 12C shows how an inflator 330 coupled to an inflation tube 332 may be used to expand or inflate the sealing elements 326 to help form or adjust the seal.

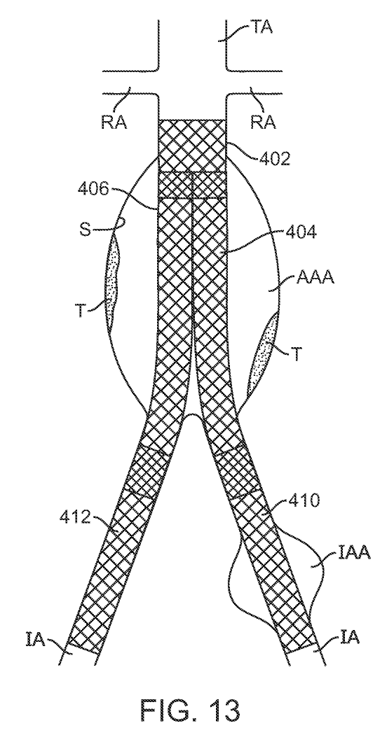

In some embodiments, additional scaffolding legs may be provided. FIG. 13 shows a docking scaffold system similar to those previously described. Docking station 402 is generally similar to scaffolds 106, 210, and 330 as described above. Leg scaffolding 404, 406, as well as additional leg scaffolding 410 and 412 may be generally similar to any of scaffoldings 112, 116, 218, 228, 325, and 328 as described above. As shown in FIG. 13, two additional leg scaffolds 410, 412 are be provided. Additional leg scaffolds 410 and 412, traverse the iliac arteries and couple to leg scaffolds 404 and 406 respectively. Additional leg scaffolds 410 and 412 are delivered via guidewire and subsequently expanded, for example, by self-expansion or balloon expansion. Additional leg scaffolds 410, 412 may be delivered and expanded into position before or after leg scaffolds 404, 406 are delivered. When additional leg scaffolds are delivered and expanded before leg scaffolds 404, 406, a downstream portion of the outside surface of leg scaffolds 404, 406 engages the upstream portion of the inside surface of additional leg scaffolds 410, 412. When additional leg scaffolds are delivered and expanded after leg scaffolds 404, 406, a downstream portion of the inside surface of leg scaffolds 404, 406 engages the upstream portion of the outside surface of additional leg scaffolds 410, 412. Additional leg scaffolds 410, 412 may be used to treat an iliac artery aneurysm IAA. Additional leg scaffolds 410, 412 may include a covering material such as Dacron™ or ePTFE so as to fully form a blood flow lumen through iliac arteries IA. The iliac artery aneurysm may then be filled with a hardenable filling material as described above. The hardening material may also help lock the scaffolds in position relative to the aneurysm thereby preventing future migration. Alternatively, additional leg scaffolds may include a filling structure which is filled with a hardenable material to help anchor the additional leg scaffolds in position and to help seal the aneurismal sac off from blood flow thereby forcing blood to flow through the lumens created by the scaffolds and their respective filling structures. While the embodiment of FIG. 13 shows one iliac artery aneurysm and two additional leg scaffolds, in other embodiments more than one iliac artery aneurysm may be present and different numbers of additional leg scaffolds may be provided.

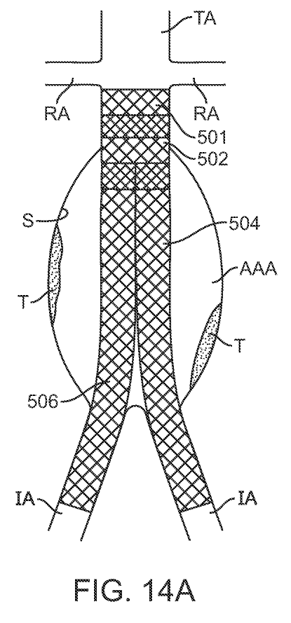

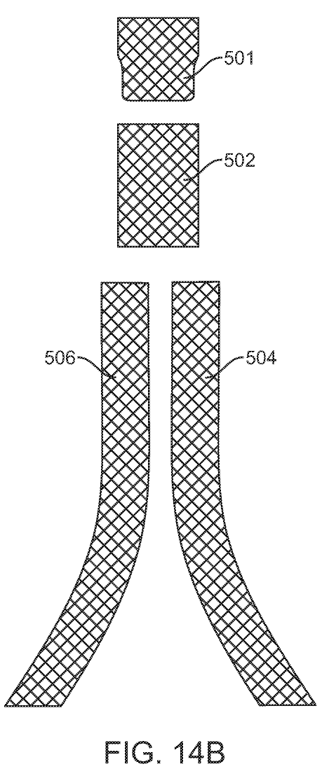

In some embodiments, a crown scaffold 501 may be provided. As shown in FIGS. 14A and 14B, crown scaffold 501 is a bare metal stent. Crown 501 is guidewire delivered to a site upstream of an aneurysm AAA and may be self-expandable or balloon expanded. Crown 501 is often a standard, generic part while docking scaffold 502 and leg scaffolds 504, 506 may be customized for the patient. Crown 501 is often delivered and expanded after docking scaffold 502 is such that the surface of the downstream portion of crown 501 is engaged with the surface of the upstream portion of docking scaffold 502. Docking scaffold 502 and leg scaffolds 504 and 506 are generally similar to the scaffolds previously described. In some cases, a filling structure may be provided for the crown scaffold to help anchor it in position relative to an aneurysm. FIG. 14A shows the crown scaffold 501, docking scaffold 502, and leg scaffolds 504 and 506 delivered and expanded in position relative to the aneurysm AAA. For clarity, FIG. 14B shows an exploded view of the expanded scaffolds.

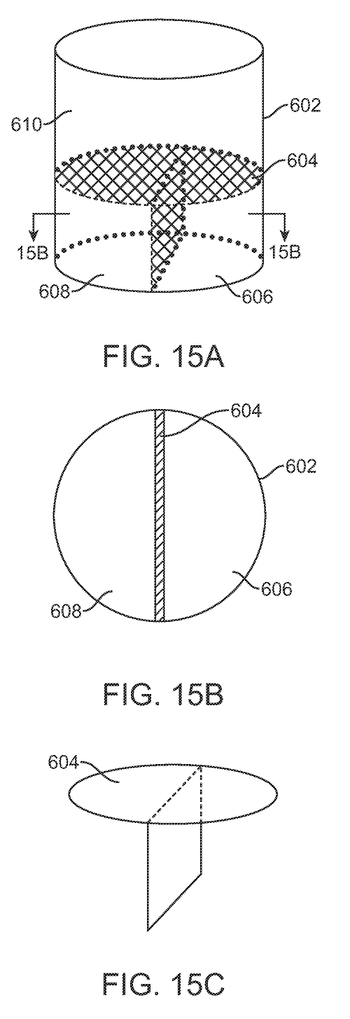

In some instances, a docking scaffold 602 may include a divider 604. Divider 604 is often integrally formed with docking scaffold 602, which is a stent-like scaffold. As shown in FIG. 15A, 602 is shown shaded. Divider 604 splits the inside volume of docking scaffold 602 into an upstream portion 610 with a circular cross section, and two downstream portions 606 and 608 with D-shaped cross sections as shown in FIG. 15B. When leg scaffolds are delivered and expanded within the downstream portions of scaffold 602, divider 604 keeps the leg scaffolds from taking more cross-sectional area than allotted. Divider 604 also prevents the leg scaffolds from intruding too far upstream into the central passageway of docking scaffold 602. For clarity, divider 604 is shown without the rest of docking scaffold 602 in FIG. 15C.

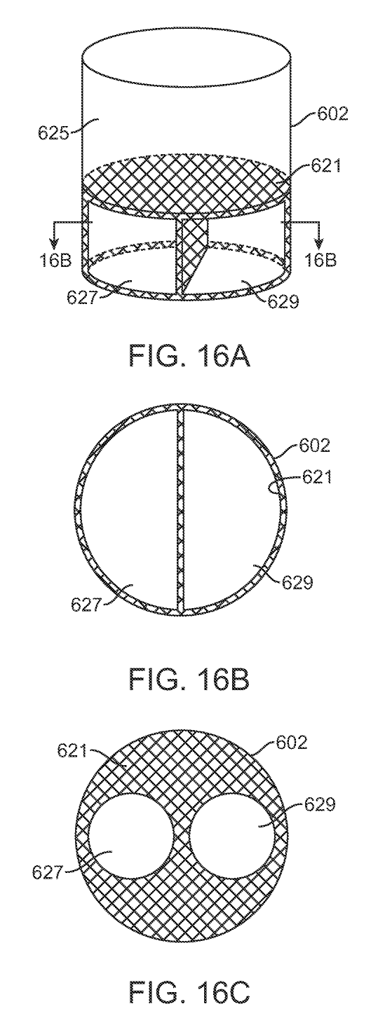

An internal double-walled filling structure 621 may also be used as a divider. As seen in FIG. 16A, filling structure or divider 621 splits the inside volume of docking scaffold 621 into upstream portion 625 with a circular cross section and two downstream portions 627 and 629. After leg scaffolds are delivered and expanded within the downstream portions 627 and 629, divider 621 can be filled and expanded such that it holds the leg scaffolds in place. FIGS. 16A and 16B show divider 621 unfilled. FIG. 16C shows divider 621 when filled.

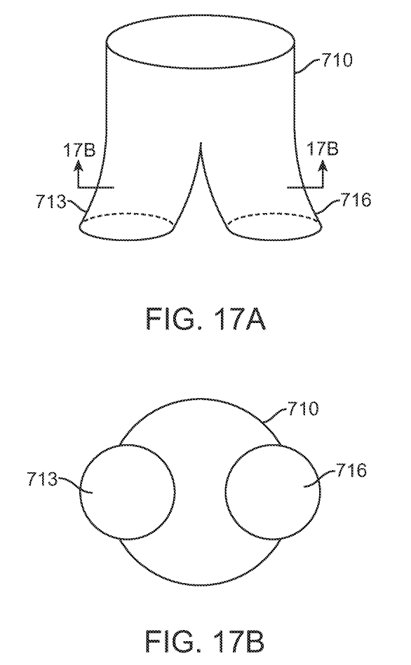

The docking scaffold may also be formed so that the leg scaffolds are prevented from intruding on one another. As seen in FIGS. 17A and 17B, the downstream portion of docking scaffold 710 bifurcates into a first portion 713 and a second portion 716. Each portion 713, 716 has its own, generally circular lumen for receiving a leg scaffold. Double-layered filling structures may also be provided for docking scaffold 710, docking scaffold portion 713, and/or docking scaffold 716 to hold the docking scaffold in place relative to an aneurysm and/or attached leg scaffolds.

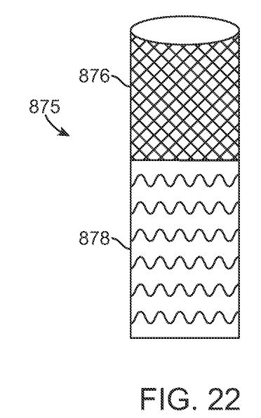

While typical scaffold structures are often either balloon expandable or self-expanding, in some embodiments it may be advantageous to provide a scaffold having a balloon expandable region and a self-expanding region. For example, FIG. 22 illustrates a scaffold 875 having an upper portion that is balloon expandable 876 and a lower portion that is self-expanding 878. In this embodiment, the two regions are illustrated as being approximately the same length, although one will appreciate that region length may be adjusted as required. In this embodiment, the self-expanding region is advantageous since it will expand until it engages the vessel wall or docking scaffold or it can expand to a predetermined shape, such as a D-shape. This is particularly desirable in situations where a physician wishes to avoid using a balloon to expand aneurismal tissue which may be damaged or significantly weakened or where it is difficult to form the desired shape by balloon expansion. A balloon expandable region is desirable when a fixed diameter is needed unlike the self-expanding scaffolds which may continue to radially expand. The balloon expandable portion 876 may be integrally formed with the self-expanding region, for example by laser cutting the stent from a Nitinol tube and then differentially heat treating the two sections, or two discrete sections may be joined together by welding, suturing, bonding, etc.

While the above is a complete description of the preferred embodiments of the invention, various alternatives, modifications, and equivalents may be used. Therefore, the above description should not be taken as limiting in scope of the invention which is defined by the appended claims.

What is claimed is:

1. A system for treating an aneurysm in a blood vessel, said system comprising:
a docking scaffold radially expandable from a contracted configuration to an expanded configuration and having an upstream end, a downstream end and a central passageway therebetween, wherein in the expanded configuration the upstream end engages a portion of the blood vessel upstream of the aneurysm;
a first leg scaffold radially expandable from a contracted configuration to an expanded configuration, wherein a portion of the first leg scaffold is slidably received in the central passageway such that an outside surface of the first leg scaffold in the expanded configuration engages an inside surface of the docking scaffold;
a second leg scaffold radially expandable from a contracted configuration to an expanded configuration, wherein a portion of the second leg scaffold is slidably received in the central passageway such that an outside surface of the second leg scaffold in the expanded configuration engages an inside surface of the docking scaffold, and
a first double-walled filling structure, the filling structure having an outer wall and an inner wall, wherein the filling structure is adapted to be filled with a hardenable fluid filling medium so that the outer wall conforms to an inside surface of the aneurysm and the inner wall forms a first substantially tubular lumen to provide a path for blood flow therethrough,
wherein the first double-walled filling structure is coupled with at least one of the leg scaffolds in the expanded configuration.

2. A system as in claim 1, wherein in the expanded configuration the outer surface of the first leg scaffold engages the outer surface of the second leg scaffold in the expanded configuration to define a mating region, wherein the mating region is disposed at least partially within the central passageway.

3. A system as in claim 2, wherein the mating region forms a generally double D-shaped cross-section.

4. A system as in claim 1, wherein the first leg and the second leg scaffolds cross each other as they traverse the aneurysm.

5. A system as in claim 1, wherein the downstream end of the docking scaffold is disposed upstream of the aneurysm.

6. A system as in claim 1, wherein the downstream end of the docking scaffold is disposed in the aneurismal sac.

7. A system as in claim 1, wherein the downstream end of the docking scaffold is disposed below the aneurysm.

8. A system as in claim 1, wherein the docking scaffold is disposed in the blood vessel so as to traverse a renal artery bifurcation without inhibiting blood flow thereto.

9. A system as in claim 1, further comprising a second double-walled filling structure, the second filling structure having an outer wall and an inner wall, wherein the second filling structure is adapted to be filled with a hardenable fluid filling medium so that the outer wall conforms to an inside surface of the aneurysm and the inner wall forms a second substantially tubular lumen to provide a path for blood flow therethrough,
wherein the second double-walled filling structure is coupled with the second leg scaffold in the expanded configuration.

10. A system as in claim 1, further comprising a third double-walled filling structure, the third filling structure having an outer wall and an inner wall, wherein the third filling structure is adapted to be filled with a hardenable fluid filling medium so that the outer wall conforms to an inside surface of the aneurysm and the inner wall forms a third substantially tubular lumen to provide a path for blood flow therethrough,
wherein the third double-walled filling structure is disposed at least partially over the docking scaffold in the expanded configuration.

11. A system as in claim 10, wherein an upstream portion of the docking scaffold remains uncovered by the third double-walled filling structure in the expanded configuration.

12. A system as in claim 11, wherein the uncovered upstream portion engages the blood vessel in the expanded configuration.

13. A system as in claim 10, wherein when filled with filling medium, the third double-walled filling structure seals an upper portion of the aneurysm thereby preventing blood flow between the outer wall of the third double-walled filling structure and an inner wall of the blood vessel.

14. A system as in claim 10, wherein a downstream portion of the docking scaffold remains uncovered by the third double-walled filling structure in the expanded configuration.

15. A system as in claim 1, wherein the docking scaffold comprises an expandable region, the expandable region adapted to linearly expand and contract.

16. A system as in claim 1, wherein the docking scaffold comprises an external flange.

17. A system as in claim 1, wherein the docking scaffold comprises a self-expanding region and a balloon expandable region.

18. A system as in claim 1, wherein the docking scaffold comprises a restraining element, the restraining element limiting expansion of at least a portion of the docking scaffold to a target diameter.

19. A system as in claim 18, wherein the restraining element comprises a band disposed around the docking scaffold.

20. A system as in claim 18, wherein the restraining element forms a tapered region on one end of the docking scaffold in the expanded configuration.

21. A system as in claim 1, wherein the docking scaffold comprises an expandable restraining element, the expandable restraining element limiting expansion of at least a portion of the docking scaffold to a target diameter.

22. A system as in claim 1, wherein an upstream portion of the first leg scaffold remains uncovered in the expanded configuration.

23. A system as in claim 1, wherein a downstream portion of the first leg scaffold remains uncovered in the expanded configuration.

24. A system as in claim 23, wherein the downstream portion of the first leg scaffold is disposed in an iliac artery.

25. A system as in claim 1, wherein an upstream portion of the second leg scaffold remains uncovered in the expanded configuration.

26. A system as in claim 1, wherein a downstream portion of the second leg scaffold remains uncovered in the expanded configuration.

27. A system as in claim 26, wherein the downstream portion of the second leg scaffold is disposed in an iliac artery.

28. A system as in claim 1, wherein at least one of the first or second leg scaffolds comprise an external flange.

29. A system as in claim 1, wherein at least one of the first or second leg scaffolds comprise a self-expanding region and a balloon expandable region.

30. A system as in claim 1, wherein the first leg scaffold comprises a sealing element disposed at least partially along the portion of the first leg scaffold slidably received in the central passageway, the sealing element forming a seal between the outside surface of the first leg scaffold in the expanded configuration and the inside surface of the docking scaffold.

31. A system as in claim 30, wherein the sealing element is expandable.

32. A system as in claim 1, wherein the second leg scaffold comprises a sealing element disposed at least partially along the portion of the second leg scaffold slidably received in the central passageway, the sealing element forming a seal between the outside surface of the second leg scaffold in the expanded configuration and the inside surface of the second leg scaffold.

33. A system as in claim 32, wherein the sealing element is expandable.

34. A system as in claim 1, further comprising a third leg scaffold radially expandable from a contracted configuration to an expanded configuration, wherein a portion of the third leg scaffold is slidably received by the first or second leg scaffold such that a surface of the third leg scaffold in the expanded configuration engages a surface of the first or second leg scaffold.

35. A system as in claim 34, wherein a portion of the third leg scaffold is slidably received by the first or second leg scaffold such that an inside surface of the third leg scaffold in the expanded configuration engages an outside surface of the first or second leg scaffold.

36. A system as in claim 34, wherein the upstream end of the third leg scaffold is disposed in an iliac artery.

37. A system as in claim 34, further comprising a fourth double-walled filling structure, the fourth filling structure having an outer wall and an inner wall, wherein the fourth filling structure is adapted to be filled with a hardenable fluid filling medium so that the outer wall conforms to an inner surface of the aneurysm and the inner wall forms a fourth substantially tubular lumen to provide a path for blood flow therethrough,
wherein the fourth double-walled filling structure is coupled with the third leg scaffold.

38. A system as in claim 34, further comprising a fourth leg scaffold radially expandable from a contracted configuration to an expanded configuration, wherein a portion of the fourth leg scaffold is slidably received by the second leg scaffold such that a surface of the fourth leg scaffold in the expanded configuration engages a surface of the second leg scaffold, and wherein an inside surface of the fourth leg scaffold in the expanded configuration engages an outside surface of the second leg scaffold.

39. A system as in claim 38, further comprising a fifth double-walled filling structure, the fifth filling structure having an outer wall and an inner wall, wherein the fifth filling structure is adapted to be filled with a hardenable fluid filling medium so that the outer wall conforms to an inner surface of the aneurysm and the inner wall forms a fifth substantially tubular lumen to provide a path for blood flow therethrough,
wherein the fifth double-walled filling structure is coupled with the fourth leg scaffold.

40. A system as in claim 1, further comprising a crown scaffold radially expandable from a contracted configuration to an expanded configuration and having an upstream portion and a downstream portion, wherein the downstream portion of the crown scaffold is slidably received by the upstream end of the docking scaffold.

41. A system as in claim 40, wherein in the expanded configuration the downstream portion of the crown scaffold is slidably received in the central passageway such that an outside surface of the crown scaffold engages an inside surface of the docking scaffold.

42. A system as in claim 1, wherein the docking scaffold comprises a divider disposed within the docking scaffold and adapted to separate the slidably received portion of the first leg scaffold and from the slidably received portion of second leg scaffold.

43. A system as in claim 1, wherein the downstream end of the docking scaffold is bifurcated into a first portion and a second portion, wherein the first portion is adapted to slidably receive the first leg and the second portion is adapted to slideably receive the second leg.

44. A method for treating an aneurysm in a blood vessel, said method comprising:
advancing a docking scaffold through the blood vessel to a position upstream of the aneurysm;
radially expanding the docking scaffold from a contracted configuration to an expanded configuration, wherein in the expanded configuration the docking scaffold engages a portion of the blood vessel upstream of the aneurysm;
advancing a first leg scaffold through the blood vessel toward the docking scaffold so that the first leg scaffold is slidably received by the docking scaffold;
radially expanding the first leg scaffold from a contracted configuration to an expanded configuration, wherein in the expanded configuration the first leg scaffold engages at least a portion of an inner surface of the docking scaffold;
advancing a second leg scaffold through the blood vessel toward the docking scaffold so that the second leg scaffold is slidably received by the docking scaffold;
radially expanding the second leg scaffold from a contracted configuration to an expanded configuration, wherein in the expanded configuration the second leg scaffold engages at least a portion of the inner surface of the docking scaffold;
advancing a first double-walled filling structure through the blood vessel toward the aneurysm; and
filling the first double-walled filling structure with a fluid filling medium so that an outer wall of the first filling structure conforms to an inside surface of the aneurysm and an inner wall of the first filling structure forms a first substantially tubular lumen to provide a first blood flow path across the aneurysm,
wherein the first filling structure is coupled with at least one of the leg scaffolds in the expanded configuration.

45. A method as in claim 44, wherein advancing the docking scaffold comprises positioning at least a portion of the docking scaffold upstream of the aneurysm.

46. A method as in claim 44, wherein advancing the docking scaffold comprises positioning at least a portion of the docking scaffold across the aneurysm.

47. A method as in claim 44, wherein advancing the docking scaffold comprises positioning at least a portion of the docking scaffold downstream of the aneurysm.

48. A method as in claim 44, wherein advancing the docking scaffold comprises positioning at least a portion of the docking scaffold across a renal artery bifurcation without obstructing blood flow into the renal artery.

49. A method as in claim 44, further comprising restraining a portion of the docking scaffold during radial expansion.

50. A method as in claim 49, wherein restraining a portion of the docking scaffold forms a region of the docking scaffold having a constant predetermined diameter.

51. A method as in claim 49, wherein restraining a portion of the docking scaffold forms a tapered region.

52. A method as in claim 49, wherein restraining comprises limiting radial expansion of the docking scaffold with a band disposed circumferentially therearound.

53. A method as in claim 44, wherein radially expanding the first leg scaffold and second leg scaffold to the expanded configuration comprises engaging the first leg scaffold with the second leg scaffold.

54. A method as in claim 44, wherein advancing the first leg scaffold and second leg scaffold comprises crossing the first leg scaffold with the second leg scaffold.

55. A method as in claim 44, further comprising advancing a second double-walled filling structure through the blood vessel toward the aneurysm.

56. A method as in claim 55, further comprising filling the second double-walled filling structure with a fluid filling medium so that an outer wall of the second filling structure conforms to an inside surface of the aneurysm and an inner wall of the second filling structure forms a second substantially tubular lumen to provide a second blood flow path across the aneurysm,
wherein the second filling structure is disposed at least partially over the second leg scaffold in the expanded configuration.

57. A method as in claim 44, further comprising advancing a third double-walled filling structure through the blood vessel toward the aneurysm.

58. A method as in claim 44, wherein advancing the first leg scaffold comprises positioning a portion of the first leg scaffold in an iliac artery.

59. A method as in claim 44, wherein advancing the second leg scaffold comprises positioning a portion of the second leg scaffold in an iliac artery.

60. A method as in claim 44, further comprising sealing the first leg and the second leg scaffolds within the docking scaffold to prevent blood flow between an outer surface of the first leg and second leg scaffolds and an inner surface of the docking scaffold.

61. A method as in claim 60, wherein sealing comprises inflating a sealing element.

62. A method as in claim 44, further comprising advancing a third leg scaffold through the blood vessel toward the first or second leg scaffold so that the third leg scaffold is slidably received by the first or second leg scaffold; and
radially expanding the third leg scaffold from a contracted configuration to an expanded configuration, wherein in the expanded configuration the third leg scaffold engages at least a portion of a surface of the first or second leg scaffold.

63. A method as in claim 62, wherein in the expanded configuration the third leg scaffold engages at least a portion of the outside surface of the first or second leg scaffold.

64. A method as in claim 62, further comprising advancing a fourth double-walled filling structure with a fluid filling medium so that an outer wall of the fourth filling structure conforms to an inside surface of the aneurysm and an inner wall of the fourth filling structure forms a fourth substantially tubular lumen to provide a fourth blood flow path.

65. A method as in claim 64, wherein the fourth filling structure is disposed at least partially over the third leg scaffold in the expanded configuration.

66. A method as in claim 62, further comprising advancing a fourth leg scaffold through the blood vessel towards the second leg scaffold so that the fourth leg scaffold is slidably received by the second leg scaffold; and
radially expanding the fourth leg scaffold from a contracted configuration to an expanded configuration, wherein in the expanded configuration the fourth leg scaffold engages at least a portion of a surface of the second leg scaffold.

67. A method as in claim 66, wherein in the expanded configuration the fourth leg scaffold engages at least a portion of the outside surface of the second leg scaffold.

68. A method as in claim 66, further comprising advancing a fifth double-walled filling structure with a fluid filling medium so that an outer wall of the fifth filling structure conforms to an inside surface of the aneurysm and an inner wall of the fifth filling structure forms a fifth substantially tubular lumen to provide a fifth blood flow path.

69. A method as in claim 68, wherein the fifth filling structure is disposed at least partially over the fourth leg scaffold in the expanded configuration.

70. A method as in claim 44, further comprising:
advancing a crown scaffold through the blood vessel to a position upstream of the aneurysm; and
radially expanding the crown scaffold from a contracted configuration to an expanded configuration, wherein in the expanded configuration the crown scaffold engages the upstream end of the docking scaffold.
